SEQUENCE LISTING (8 sequences)

SEQ ID NO: 1 - DNA, 2988 bp, Artificial Sequence
  2.2 1L1L construct
atggcgtccgcagcaccactggtcacggcaatgtgtttgctcggaaatgtgagcttcccatgcgaccgcccgcccacatgctatacccgcgaaccttccagagccctcgacatccttgaagagaacgtgaaccatgaggcctacgataccctgctcaatgccatattgcggtgcggatcgtctggcagcgtcattgacgactttaccctgaccagcccctacttgggcacatgctcgtactgccaccatactgaaccgtgcttcagccctgttaagatcgagcaggtctgggacgaagcggacgataacaccatacgcatacagacttccgcccagtttggatacgaccaaagcggagcagcaagcgcaaacaagtaccgctacatggcggctgcggcggtaacaggcggaggaggcagcctaggggtaaccggaggcggcggatccgtaaccaccgttaaagaaggcaccatggatgacatcaagattagcacctcaggaccgtgtagaaggcttagctacaaaggatactttctcctcgcaaaatgccctccaggggacagcgtaacggttagcatagtgagtagcaactcagcaacgtcatgtacactggcccgcaagataaaaccaaaattcgtgggacgggaaaaatatgatctacctcccgttcacggtaaaaaaattccttgcacagtgtacgaccgtctggcagcaacaactgcaggctacatcactatgcacaggccgggaccgcacgcttatacatcctacctggaagaatcatcagggaaagtttacgcaaagccgccatctgggaagaacattacgtatgagtgcaagtgcggcgactacaagaccggaaccgtttcgacccgcaccgaaatcactggttgcaccgccatcaagcagtgcgtcgcctataagagcgaccaaacgaagtgggtcttcaactcaccggacttgatcagacatgacgaccacacggtccaagggaaattgcatttgcctttcaagttgatcccgagtacctgcatggtccctgttgcccacgcgccgaatgtaatacatggctttaaacacatcagcctccaattagatacagaccacttgacattgctcaccaccaggagactaggggcaaacccggaaccaaccactgaatggatcgtcggaaagacggtcagaaacttcaccgtcgaccgagatggcctggaatacatatggggaaatcatgagccagtgagggtctatgcccaagagtcagcaccaggagaccctcacggatggccacacgaaatagtacagcattactaccatcgccatcctgtgtacaccatcttagccgtcgcatcagctaccgtggcgatgatgattggcgtaactgttgcagtgttatgtgcctgtaaagcgcgccgtgagtgcctgacgccatacgccctggccccaaacgccgtaatcccaacttcgctggcactcttgtgctgcgttaggtcggccaatgctgaaacgttcaccgagaccatgagttacttgtggtcgaacagtcagccgttcttctgggtccagttgtgcatacctttggccgctttcatcgttctaatgcgctgctgctcctgctgcctgccttttttagtggttgccggcgcctacctggcgaaggtagacgcctacgaacatgcgaccactgttccaaatgtgccacagataccgtataaggcacttgttgaaagggcagggtatgccccgctcaatttggagatcactgtcatgtcctcggaggttttgccttccaccaaccaagagtacattacctgcaaattcaccactgtggtcccctccccaaaaatcaaatgctgcggctccttggaatgtcagccggccgctcatgcaggctatacctgcaaggtcttcggaggggtctacccctttatgtggggaggagcgcaatgtttttgcgacagtgagaacagccagatgagtgaggcgtacgtcgaattgtcagcagattgcgcgtctgaccacgcgcaggcgattaaggtgcacactgccgcgatgaaagtaggactgcgtattgtgtacgggaacactaccagtttcctagatgtgtacgtgaacggagtcacaccaggaacgtctaaagacttgaaagtcatagctggaccaatttcagcatcgtttacgccattcgatcataaggtcgttatccatcgcggcctggtgtacaactatgacttcccggaatatggagcgatgaaaccaggagcgtttggagacattcaagctacctccttgactagcaaggatctcatcgccagcacagacattaggctactcaagccttcctcaggaaacgtgcatgtcccgtacacgcaggcctcatcaggatttgagatgtggaaaaacaactcaggccgcccactgcaggaaaccgcacctttcgggtgtaagattgcagtaaatccgctccgagcggtggactgttcatacgggaacattcccatttctattgacatcccgaacgctgcctttatcaggacatcagatgcaccactggtctcaacagtcaaatgtgaagtcagtgagtgcacttattcagcagacttcggcgggatggccaccctgcagtatgtatccgaccgcgaaggtcaatgccccgtacattcgcattcgagcacagcaactctccaagagtcgacagtacatgtcctggagaaaggagcggtgacagtacactttagcaccgcgagtccacaggcgaactttatcgtatcgctgtgtgggaagaagacaacatgcaatgcagaatgtaaaccaccagctgaccatatcgtgagcaccccgcacaaaaatgaccaagaatttcaagccgccatctcaaaaacatcatggagttggctgtttgcccttttcggcggcgcctcgtcgctattaattataggacttatgatttttgcttgcagcatgatgctgactagcacacgaagatga

SEQ ID NO: 2 - protein, 995 aa, Artificial Sequence
  2.2 1L1L construct
MASAAPLVTAMCLLGNVSFPCDRPPTCYTREPSRALDILEENVNHEAYDTLLNAILRCGSSGSVIDDFTLTSPYLGTCSYCHHTEPCFSPVKIEQVWDEADDNTIRIQTSAQFGYDQSGAASANKYRYMAAAAVTGGGGSLGVTGGGGSVTTVKEGTMDDIKISTSGPCRRLSYKGYFLLAKCPPGDSVTVSIVSSNSATSCTLARKIKPKFVGREKYDLPPVHGKKIPCTVYDRLAATTAGYITMHRPGPHAYTSYLEESSGKVYAKPPSGKNITYECKCGDYKTGTVSTRTEITGCTAIKQCVAYKSDQTKWVFNSPDLIRHDDHTVQGKLHLPFKLIPSTCMVPVAHAPNVIHGFKHISLQLDTDHLTLLTTRRLGANPEPTTEWIVGKTVRNFTVDRDGLEYIWGNHEPVRVYAQESAPGDPHGWPHEIVQHYYHRHPVYTILAVASATVAMMIGVTVAVLCACKARRECLTPYALAPNAVIPTSLALLCCVRSANAETFTETMSYLWSNSQPFFWVQLCIPLAAFIVLMRCCSCCLPFLVVAGAYLAKVDAYEHATTVPNVPQIPYKALVERAGYAPLNLEITVMSSEVLPSTNQEYITCKFTTVVPSPKIKCCGSLECQPAAHAGYTCKVFGGVYPFMWGGAQCFCDSENSQMSEAYVELSADCASDHAQAIKVHTAAMKVGLRIVYGNTTSFLDVYVNGVTPGTSKDLKVIAGPISASFTPFDHKVVIHRGLVYNYDFPEYGAMKPGAFGDIQATSLTSKDLIASTDIRLLKPSSGNVHVPYTQASSGFEMWKNNSGRPLQETAPFGCKIAVNPLRAVDCSYGNIPISIDIPNAAFIRTSDAPLVSTVKCEVSECTYSADFGGMATLQYVSDREGQCPVHSHSSTATLQESTVHVLEKGAVTVHFSTASPQANFIVSLCGKKTTCNAECKPPADHIVSTPHKNDQEFQAAISKTSWSWLFALFGGASSLLIIGLMIFACSMMLTSTRR

SEQ ID NO: 3 - DNA, 3327 bp, Artificial Sequence
  2.2 construct
atggcgtccgcagcaccactggtcacggcaatgtgtttgctcggaaatgtgagcttcccatgcgaccgcccgcccacatgctatacccgcgaaccttccagagccctcgacatccttgaagagaacgtgaaccatgaggcctacgataccctgctcaatgccatattgcggtgcggatcgtctggcagcgtcattgacgactttaccctgaccagcccctacttgggcacatgctcgtactgccaccatactgaaccgtgcttcagccctgttaagatcgagcaggtctgggacgaagcggacgataacaccatacgcatacagacttccgcccagtttggatacgaccaaagcggagcagcaagcgcaaacaagtaccgctacatggcggctgcggcggtaaccgacaacaaattcaacaaagaacaacaaaacgcgttctatgagatcttacatttacctaacttaaacgaagaacaacgaaacgccttcatccaaagtttaaaagatgacccaagccaaagcgctaaccttttagcagaagctaaaaagctaaatgatgctcaggcgccgaaagtagacaacaaattcaacaaagaacaacaaaacgcgttctatgagatcttacatttacctaacttaaacgaagaacaacgaaacgccttcatccaaagtttaaaagatgacccaagccaaagcgctaaccttttagcagaagctaaaaagctaaatgatgctcaggcgccgaaagtagacgcgaattcgagctcggtacccggggatccggtaaccaccgttaaagaaggcaccatggatgacatcaagattagcacctcaggaccgtgtagaaggcttagctacaaaggatactttctcctcgcaaaatgccctccaggggacagcgtaacggttagcatagtgagtagcaactcagcaacgtcatgtacactggcccgcaagataaaaccaaaattcgtgggacgggaaaaatatgatctacctcccgttcacggtaaaaaaattccttgcacagtgtacgaccgtctggcagcaacaactgcaggctacatcactatgcacaggccgggaccgcacgcttatacatcctacctggaagaatcatcagggaaagtttacgcaaagccgccatctgggaagaacattacgtatgagtgcaagtgcggcgactacaagaccggaaccgtttcgacccgcaccgaaatcactggttgcaccgccatcaagcagtgcgtcgcctataagagcgaccaaacgaagtgggtcttcaactcaccggacttgatcagacatgacgaccacacggtccaagggaaattgcatttgcctttcaagttgatcccgagtacctgcatggtccctgttgcccacgcgccgaatgtaatacatggctttaaacacatcagcctccaattagatacagaccacttgacattgctcaccaccaggagactaggggcaaacccggaaccaaccactgaatggatcgtcggaaagacggtcagaaacttcaccgtcgaccgagatggcctggaatacatatggggaaatcatgagccagtgagggtctatgcccaagagtcagcaccaggagaccctcacggatggccacacgaaatagtacagcattactaccatcgccatcctgtgtacaccatcttagccgtcgcatcagctaccgtggcgatgatgattggcgtaactgttgcagtgttatgtgcctgtaaagcgcgccgtgagtgcctgacgccatacgccctggccccaaacgccgtaatcccaacttcgctggcactcttgtgctgcgttaggtcggccaatgctgaaacgttcaccgagaccatgagttacttgtggtcgaacagtcagccgttcttctgggtccagttgtgcatacctttggccgctttcatcgttctaatgcgctgctgctcctgctgcctgccttttttagtggttgccggcgcctacctggcgaaggtagacgcctacgaacatgcgaccactgttccaaatgtgccacagataccgtataaggcacttgttgaaagggcagggtatgccccgctcaatttggagatcactgtcatgtcctcggaggttttgccttccaccaaccaagagtacattacctgcaaattcaccactgtggtcccctccccaaaaatcaaatgctgcggctccttggaatgtcagccggccgctcatgcaggctatacctgcaaggtcttcggaggggtctacccctttatgtggggaggagcgcaatgtttttgcgacagtgagaacagccagatgagtgaggcgtacgtcgaattgtcagcagattgcgcgtctgaccacgcgcaggcgattaaggtgcacactgccgcgatgaaagtaggactgcgtattgtgtacgggaacactaccagtttcctagatgtgtacgtgaacggagtcacaccaggaacgtctaaagacttgaaagtcatagctggaccaatttcagcatcgtttacgccattcgatcataaggtcgttatccatcgcggcctggtgtacaactatgacttcccggaatatggagcgatgaaaccaggagcgtttggagacattcaagctacctccttgactagcaaggatctcatcgccagcacagacattaggctactcaagccttcctcaggaaacgtgcatgtcccgtacacgcaggcctcatcaggatttgagatgtggaaaaacaactcaggccgcccactgcaggaaaccgcacctttcgggtgtaagattgcagtaaatccgctccgagcggtggactgttcatacgggaacattcccatttctattgacatcccgaacgctgcctttatcaggacatcagatgcaccactggtctcaacagtcaaatgtgaagtcagtgagtgcacttattcagcagacttcggcgggatggccaccctgcagtatgtatccgaccgcgaaggtcaatgccccgtacattcgcattcgagcacagcaactctccaagagtcgacagtacatgtcctggagaaaggagcggtgacagtacactttagcaccgcgagtccacaggcgaactttatcgtatcgctgtgtgggaagaagacaacatgcaatgcagaatgtaaaccaccagctgaccatatcgtgagcaccccgcacaaaaatgaccaagaatttcaagccgccatctcaaaaacatcatggagttggctgtttgcccttttcggcggcgcctcgtcgctattaattataggacttatgatttttgcttgcagcatgatgctgactagcacacgaagatga

SEQ ID NO: 4 - protein, 1108 aa, Artificial Sequence
  2.2 vector construct
MASAAPLVTAMCLLGNVSFPCDRPPTCYTREPSRALDILEENVNHEAYDTLLNAILRCGSSGSVIDDFTLTSPYLGTCSYCHHTEPCFSPVKIEQVWDEADDNTIRIQTSAQFGYDQSGAASANKYRYMAAAAVTDNKFNKEQQNAFYEILHLPNLNEEQRNAFIQSLKDDPSQSANLLAEAKKLNDAQAPKVDNKFNKEQQNAFYEILHLPNLNEEQRNAFIQSLKDDPSQSANLLAEAKKLNDAQAPKVDANSSSVPGDPVTTVKEGTMDDIKISTSGPCRRLSYKGYFLLAKCPPGDSVTVSIVSSNSATSCTLARKIKPKFVGREKYDLPPVHGKKIPCTVYDRLAATTAGYITMHRPGPHAYTSYLEESSGKVYAKPPSGKNITYECKCGDYKTGTVSTRTEITGCTAIKQCVAYKSDQTKWVFNSPDLIRHDDHTVQGKLHLPFKLIPSTCMVPVAHAPNVIHGFKHISLQLDTDHLTLLTTRRLGANPEPTTEWIVGKTVRNFTVDRDGLEYIWGNHEPVRVYAQESAPGDPHGWPHEIVQHYYHRHPVYTILAVASATVAMMIGVTVAVLCACKARRECLTPYALAPNAVIPTSLALLCCVRSANAETFTETMSYLWSNSQPFFWVQLCIPLAAFIVLMRCCSCCLPFLVVAGAYLAKVDAYEHATTVPNVPQIPYKALVERAGYAPLNLEITVMSSEVLPSTNQEYITCKFTTVVPSPKIKCCGSLECQPAAHAGYTCKVFGGVYPFMWGGAQCFCDSENSQMSEAYVELSADCASDHAQAIKVHTAAMKVGLRIVYGNTTSFLDVYVNGVTPGTSKDLKVIAGPISASFTPFDHKVVIHRGLVYNYDFPEYGAMKPGAFGDIQATSLTSKDLIASTDIRLLKPSSGNVHVPYTQASSGFEMWKNNSGRPLQETAPFGCKIAVNPLRAVDCSYGNIPISIDIPNAAFIRTSDAPLVSTVKCEVSECTYSADFGGMATLQYVSDREGQCPVHSHSSTATLQESTVHVLEKGAVTVHFSTASPQANFIVSLCGKKTTCNAECKPPADHIVSTPHKNDQEFQAAISKTSWSWLFALFGGASSLLIIGLMIFACSMMLTSTRR

SEQ ID NO: 5 - DNA, 3567 bp, Artificial Sequence
  E2 71 eMA vector construct
atggcgtccgcagcaccactggtcacggcaatgtgtttgctcggaaatgtgagcttcccatgcgaccgcccgcccacatgctatacccgcgaaccttccagagccctcgacatccttgaagagaacgtgaaccatgaggcctacgataccctgctcaatgccatattgcggtgcggatcgtctggcagcgtcattgacgactttaccctgaccagcccctacttgggcacatgctcgtactgccaccatactgaaccgtgcttcagccctgttaagatcgagcaggtctgggacgaagcggacgataacaccatacgcatacagacttccgcccagtttggatacgaccaaagcggagcagcaagcgcaaacaagtaccgctacatggcggctgcggcggtaacaggcggaggaggcagcctaggcgtgaccagcagcggctctggatctggcggaggcggaggaagtggagggggaggaagcggcggagggggctctgctagcttcgacgcctccaatttcaaggacttcagcagcatcgcctctgcctctagctcctggcagaaccagcacggcagcaccatgatcatccaggtggactcttttggcaatgtgagcggacagtacgtgaacagggccgagggcacaggatgccagaattccccctatcctctgaccggcagagtgaacggcaccttcatcgacttcagcgtgaagtggaacaattctaccgagaactgtaatagcaacacccagtggacaggctacgcccaagtgaatggcaacaataccgagatcgtgacacggtggaacctgaagtacgagggaggatctggaccagcaatctggcagggccaggacacattccagtatgtgcccaccacagaaatcgatgctggcgggggaggctctggaagtggatctgggggtggcggcggaagctctggatctggaagcggagggggaggcagcggcagtggatcaggggggggagggggatctctggtaaccggaggcggcggatccgtaaccaccgttaaagaaggcaccatggatgacatcaagattagcacctcaggaccgtgtagaaggcttagctacaaaggatactttctcctcgcaaaatgccctccaggggacagcgtaacggttagcatagtgagtagcaactcagcaacgtcatgtacactggcccgcaagataaaaccaaaattcgtgggacgggaaaaatatgatctacctcccgttcacggtaaaaaaattccttgcacagtgtacgaccgtctggcagcaacaactgcaggctacatcactatgcacaggccgggaccgcacgcttatacatcctacctggaagaatcatcagggaaagtttacgcaaagccgccatctgggaagaacattacgtatgagtgcaagtgcggcgactacaagaccggaaccgtttcgacccgcaccgaaatcactggttgcaccgccatcaagcagtgcgtcgcctataagagcgaccaaacgaagtgggtcttcaactcaccggacttgatcagacatgacgaccacacggtccaagggaaattgcatttgcctttcaagttgatcccgagtacctgcatggtccctgttgcccacgcgccgaatgtaatacatggctttaaacacatcagcctccaattagatacagaccacttgacattgctcaccaccaggagactaggggcaaacccggaaccaaccactgaatggatcgtcggaaagacggtcagaaacttcaccgtcgaccgagatggcctggaatacatatggggaaatcatgagccagtgagggtctatgcccaagagtcagcaccaggagaccctcacggatggccacacgaaatagtacagcattactaccatcgccatcctgtgtacaccatcttagccgtcgcatcagctaccgtggcgatgatgattggcgtaactgttgcagtgttatgtgcctgtaaagcgcgccgtgagtgcctgacgccatacgccctggccccaaacgccgtaatcccaacttcgctggcactcttgtgctgcgttaggtcggccaatgctgaaacgttcaccgagaccatgagttacttgtggtcgaacagtcagccgttcttctgggtccagttgtgcatacctttggccgctttcatcgttctaatgcgctgctgctcctgctgcctgccttttttagtggttgccggcgcctacctggcgaaggtagacgcctacgaacatgcgaccactgttccaaatgtgccacagataccgtataaggcacttgttgaaagggcagggtatgccccgctcaatttggagatcactgtcatgtcctcggaggttttgccttccaccaaccaagagtacattacctgcaaattcaccactgtggtcccctccccaaaaatcaaatgctgcggctccttggaatgtcagccggccgctcatgcaggctatacctgcaaggtcttcggaggggtctacccctttatgtggggaggagcgcaatgtttttgcgacagtgagaacagccagatgagtgaggcgtacgtcgaattgtcagcagattgcgcgtctgaccacgcgcaggcgattaaggtgcacactgccgcgatgaaagtaggactgcgtattgtgtacgggaacactaccagtttcctagatgtgtacgtgaacggagtcacaccaggaacgtctaaagacttgaaagtcatagctggaccaatttcagcatcgtttacgccattcgatcataaggtcgttatccatcgcggcctggtgtacaactatgacttcccggaatatggagcgatgaaaccaggagcgtttggagacattcaagctacctccttgactagcaaggatctcatcgccagcacagacattaggctactcaagccttcctcaggaaacgtgcatgtcccgtacacgcaggcctcatcaggatttgagatgtggaaaaacaactcaggccgcccactgcaggaaaccgcacctttcgggtgtaagattgcagtaaatccgctccgagcggtggactgttcatacgggaacattcccatttctattgacatcccgaacgctgcctttatcaggacatcagatgcaccactggtctcaacagtcaaatgtgaagtcagtgagtgcacttattcagcagacttcggcgggatggccaccctgcagtatgtatccgaccgcgaaggtcaatgccccgtacattcgcattcgagcacagcaactctccaagagtcgacagtacatgtcctggagaaaggagcggtgacagtacactttagcaccgcgagtccacaggcgaactttatcgtatcgctgtgtgggaagaagacaacatgcaatgcagaatgtaaaccaccagctgaccatatcgtgagcaccccgcacaaaaatgaccaagaatttcaagccgccatctcaaaaacatcatggagttggctgtttgcccttttcggcggcgcctcgtcgctattaattataggacttatgatttttgcttgcagcatgatgctgactagcacacgaagatga

SEQ ID NO: 6 - protein, 1188 aa, Artificial Sequence
  vector construct
MASAAPLVTAMCLLGNVSFPCDRPPTCYTREPSRALDILEENVNHEAYDTLLNAILRCGSSGSVIDDFTLTSPYLGTCSYCHHTEPCFSPVKIEQVWDEADDNTIRIQTSAQFGYDQSGAASANKYRYMAAAAVTGGGGSLGVTSSGSGSGGGGGSGGGGSGGGGSASFDASNFKDFSSIASASSSWQNQHGSTMIIQVDSFGNVSGQYVNRAEGTGCQNSPYPLTGRVNGTFIDFSVKWNNSTENCNSNTQWTGYAQVNGNNTEIVTRWNLKYEGGSGPAIWQGQDTFQYVPTTEIDAGGGGSGSGSGGGGGSSGSGSGGGGSGSGSGGGGGSLVTGGGGSVTTVKEGTMDDIKISTSGPCRRLSYKGYFLLAKCPPGDSVTVSIVSSNSATSCTLARKIKPKFVGREKYDLPPVHGKKIPCTVYDRLAATTAGYITMHRPGPHAYTSYLEESSGKVYAKPPSGKNITYECKCGDYKTGTVSTRTEITGCTAIKQCVAYKSDQTKWVFNSPDLIRHDDHTVQGKLHLPFKLIPSTCMVPVAHAPNVIHGFKHISLQLDTDHLTLLTTRRLGANPEPTTEWIVGKTVRNFTVDRDGLEYIWGNHEPVRVYAQESAPGDPHGWPHEIVQHYYHRHPVYTILAVASATVAMMIGVTVAVLCACKARRECLTPYALAPNAVIPTSLALLCCVRSANAETFTETMSYLWSNSQPFFWVQLCIPLAAFIVLMRCCSCCLPFLVVAGAYLAKVDAYEHATTVPNVPQIPYKALVERAGYAPLNLEITVMSSEVLPSTNQEYITCKFTTVVPSPKIKCCGSLECQPAAHAGYTCKVFGGVYPFMWGGAQCFCDSENSQMSEAYVELSADCASDHAQAIKVHTAAMKVGLRIVYGNTTSFLDVYVNGVTPGTSKDLKVIAGPISASFTPFDHKVVIHRGLVYNYDFPEYGAMKPGAFGDIQATSLTSKDLIASTDIRLLKPSSGNVHVPYTQASSGFEMWKNNSGRPLQETAPFGCKIAVNPLRAVDCSYGNIPISIDIPNAAFIRTSDAPLVSTVKCEVSECTYSADFGGMATLQYVSDREGQCPVHSHSSTATLQESTVHVLEKGAVTVHFSTASPQANFIVSLCGKKTTCNAECKPPADHIVSTPHKNDQEFQAAISKTSWSWLFALFGGASSLLIIGLMIFACSMMLTSTRR

SEQ ID NO: 7 - DNA, 3525 bp, Artificial Sequence
  vector construct
atggcgtccgcagcaccactggtcacggcaatgtgtttgctcggaaatgtgagcttcccatgcgaccgcccgcccacatgctatacccgcgaaccttccagagccctcgacatccttgaagagaacgtgaaccatgaggcctacgataccctgctcaatgccatattgcggtgcggatcgtctggcagcgtcattgacgactttaccctgaccagcccctacttgggcacatgctcgtactgccaccatactgaaccgtgcttcagccctgttaagatcgagcaggtctgggacgaagcggacgataacaccatacgcatacagacttccgcccagtttggatacgaccaaagcggagcagcaagcgcaaacaagtaccgctacatggcggctgcggcggtaacaggcggaggaggcagcctaggcgtgaccagcagcggctctggatctggcggaggcggaggaagtggagggggaggaagcggcggagggggctctgctagcgccgaggccggcatcaccggcacatggtacaaccagcacggctctaccttcacagtgaccgcaggagcagacggaaacctgacaggacagtatgagaatagggcacagggaaccggatgccagaacagcccctacacactgaccggcaggtataatggcacaaagctggagtggcgcgtggagtggaacaatagcacagagaattgtcactcccggaccgagtggagaggacagtaccagggaggagccgaggccaggatcaacacacagtggaatctgacctatgagggaggaagcggacctgcaaccgagcagggccaggataccttcaccaaggtgaaaatcgatgctggcgggggaggctctggaagtggatctgggggtggcggcggaagctctggatctggaagcggagggggaggcagcggcagtggatcaggggggggagggggatctctggtaaccggaggcggcggatccgtaaccaccgttaaagaaggcaccatggatgacatcaagattagcacctcaggaccgtgtagaaggcttagctacaaaggatactttctcctcgcaaaatgccctccaggggacagcgtaacggttagcatagtgagtagcaactcagcaacgtcatgtacactggcccgcaagataaaaccaaaattcgtgggacgggaaaaatatgatctacctcccgttcacggtaaaaaaattccttgcacagtgtacgaccgtctggcagcaacaactgcaggctacatcactatgcacaggccgggaccgcacgcttatacatcctacctggaagaatcatcagggaaagtttacgcaaagccgccatctgggaagaacattacgtatgagtgcaagtgcggcgactacaagaccggaaccgtttcgacccgcaccgaaatcactggttgcaccgccatcaagcagtgcgtcgcctataagagcgaccaaacgaagtgggtcttcaactcaccggacttgatcagacatgacgaccacacggtccaagggaaattgcatttgcctttcaagttgatcccgagtacctgcatggtccctgttgcccacgcgccgaatgtaatacatggctttaaacacatcagcctccaattagatacagaccacttgacattgctcaccaccaggagactaggggcaaacccggaaccaaccactgaatggatcgtcggaaagacggtcagaaacttcaccgtcgaccgagatggcctggaatacatatggggaaatcatgagccagtgagggtctatgcccaagagtcagcaccaggagaccctcacggatggccacacgaaatagtacagcattactaccatcgccatcctgtgtacaccatcttagccgtcgcatcagctaccgtggcgatgatgattggcgtaactgttgcagtgttatgtgcctgtaaagcgcgccgtgagtgcctgacgccatacgccctggccccaaacgccgtaatcccaacttcgctggcactcttgtgctgcgttaggtcggccaatgctgaaacgttcaccgagaccatgagttacttgtggtcgaacagtcagccgttcttctgggtccagttgtgcatacctttggccgctttcatcgttctaatgcgctgctgctcctgctgcctgccttttttagtggttgccggcgcctacctggcgaaggtagacgcctacgaacatgcgaccactgttccaaatgtgccacagataccgtataaggcacttgttgaaagggcagggtatgccccgctcaatttggagatcactgtcatgtcctcggaggttttgccttccaccaaccaagagtacattacctgcaaattcaccactgtggtcccctccccaaaaatcaaatgctgcggctccttggaatgtcagccggccgctcatgcaggctatacctgcaaggtcttcggaggggtctacccctttatgtggggaggagcgcaatgtttttgcgacagtgagaacagccagatgagtgaggcgtacgtcgaattgtcagcagattgcgcgtctgaccacgcgcaggcgattaaggtgcacactgccgcgatgaaagtaggactgcgtattgtgtacgggaacactaccagtttcctagatgtgtacgtgaacggagtcacaccaggaacgtctaaagacttgaaagtcatagctggaccaatttcagcatcgtttacgccattcgatcataaggtcgttatccatcgcggcctggtgtacaactatgacttcccggaatatggagcgatgaaaccaggagcgtttggagacattcaagctacctccttgactagcaaggatctcatcgccagcacagacattaggctactcaagccttcctcaggaaacgtgcatgtcccgtacacgcaggcctcatcaggatttgagatgtggaaaaacaactcaggccgcccactgcaggaaaccgcacctttcgggtgtaagattgcagtaaatccgctccgagcggtggactgttcatacgggaacattcccatttctattgacatcccgaacgctgcctttatcaggacatcagatgcaccactggtctcaacagtcaaatgtgaagtcagtgagtgcacttattcagcagacttcggcgggatggccaccctgcagtatgtatccgaccgcgaaggtcaatgccccgtacattcgcattcgagcacagcaactctccaagagtcgacagtacatgtcctggagaaaggagcggtgacagtacactttagcaccgcgagtccacaggcgaactttatcgtatcgctgtgtgggaagaagacaacatgcaatgcagaatgtaaaccaccagctgaccatatcgtgagcaccccgcacaaaaatgaccaagaatttcaagccgccatctcaaaaacatcatggagttggctgtttgcccttttcggcggcgcctcgtcgctattaattataggacttatgatttttgcttgcagcatgatgctgactagcacacgaagatga

SEQ ID NO: 8 - protein, 1174 aa, Artificial Sequence
  vector construct
MASAAPLVTAMCLLGNVSFPCDRPPTCYTREPSRALDILEENVNHEAYDTLLNAILRCGSSGSVIDDFTLTSPYLGTCSYCHHTEPCFSPVKIEQVWDEADDNTIRIQTSAQFGYDQSGAASANKYRYMAAAAVTGGGGSLGVTSSGSGSGGGGGSGGGGSGGGGSASAEAGITGTWYNQHGSTFTVTAGADGNLTGQYENRAQGTGCQNSPYTLTGRYNGTKLEWRVEWNNSTENCHSRTEWRGQYQGGAEARINTQWNLTYEGGSGPATEQGQDTFTKVKIDAGGGGSGSGSGGGGGSSGSGSGGGGSGSGSGGGGGSLVTGGGGSVTTVKEGTMDDIKISTSGPCRRLSYKGYFLLAKCPPGDSVTVSIVSSNSATSCTLARKIKPKFVGREKYDLPPVHGKKIPCTVYDRLAATTAGYITMHRPGPHAYTSYLEESSGKVYAKPPSGKNITYECKCGDYKTGTVSTRTEITGCTAIKQCVAYKSDQTKWVFNSPDLIRHDDHTVQGKLHLPFKLIPSTCMVPVAHAPNVIHGFKHISLQLDTDHLTLLTTRRLGANPEPTTEWIVGKTVRNFTVDRDGLEYIWGNHEPVRVYAQESAPGDPHGWPHEIVQHYYHRHPVYTILAVASATVAMMIGVTVAVLCACKARRECLTPYALAPNAVIPTSLALLCCVRSANAETFTETMSYLWSNSQPFFWVQLCIPLAAFIVLMRCCSCCLPFLVVAGAYLAKVDAYEHATTVPNVPQIPYKALVERAGYAPLNLEITVMSSEVLPSTNQEYITCKFTTVVPSPKIKCCGSLECQPAAHAGYTCKVFGGVYPFMWGGAQCFCDSENSQMSEAYVELSADCASDHAQAIKVHTAAMKVGLRIVYGNTTSFLDVYVNGVTPGTSKDLKVIAGPISASFTPFDHKVVIHRGLVYNYDFPEYGAMKPGAFGDIQATSLTSKDLIASTDIRLLKPSSGNVHVPYTQASSGFEMWKNNSGRPLQETAPFGCKIAVNPLRAVDCSYGNIPISIDIPNAAFIRTSDAPLVSTVKCEVSECTYSADFGGMATLQYVSDREGQCPVHSHSSTATLQESTVHVLEKGAVTVHFSTASPQANFIVSLCGKKTTCNAECKPPADHIVSTPHKNDQEFQAAISKTSWSWLFALFGGASSLLIIGLMIFACSMMLTSTRR

(19) United States
(12) Patent Application Publication
(10) Pub. No.: US 2022/0025397 A1
(43) Pub. Date: Jan. 27, 2022

(54) LIGAND-DIRECTED TARGETING VECTORS

(71) Applicant: THE REGENTS OF THE UNIVERSITY OF CALIFORNIA, OAKLAND, CA (US)

(72) Inventor: Kouki MORIZONO, LOS ANGELES, CA (US)

(73) Assignee: THE REGENTS OF THE UNIVERSITY OF CALIFORNIA, OAKLAND, CA (US)

(21) Appl. No.: 17/250,921

(22) PCT Filed: Sep. 27, 2019
(86) PCT No.: PCT/US19/53686

§ 371 Date: Mar. 26, 2021

Related U.S. Application Data
(60) Provisional application No. 62/738,695, filed on Sep. 28, 2018.

(51) Int. Cl. C12N 15/86; C07K 14/005; C07K 14/195; A61K 47/68; A61K 47/64

(52) U.S. Cl. CPC C12N 15/86; C07K 14/005; C07K 14/195; A61K 47/6811; A61K 47/64; A61K 38/00; C12N 2770/36142; C12N 2810/855; C12N 2770/36122; C07K 2319/30; C12N 2740/15043

(57) ABSTRACT

Described is a targeting construct encoding a modified Sindbis virus envelope protein, comprising mutations in the E1, E2 and/or E3 proteins, fused with a monomeric biotin-binding molecule. Lentiviral vectors pseudotyped with the novel envelope proteins, such as E2 71 eMA and E2 71 mSAH, can be conjugated with biotinylated targeting ligands The conjugated ligands mediate specific binding and transduction of the target cell types. This lentiviral transduction system can be used to selectively deliver transgenes into specific cell types in vivo, which increases the numbers of vectors reaching the targeted cells and tissues and decreases adverse effects in non-targeted cells and tissues. The vectors transduce B cells without conjugation of targeting ligands. The B cell type most efficiently transduced in this manner is long-lived plasma cells, and thus can be used for long-term transgene expression.

This application claims benefit of United States provisional patent application number 62/738,695, filed Sep. 28, 2018, the entire contents of which are incorporated by reference into this application.

### STATEMENT REGARDING FEDERALLY SPONSORED RESEARCH

This invention was made with government support under Grant Number Al108400, awarded by the National Institutes of Health. The government has certain rights in the invention.

### REFERENCE TO A SEQUENCE LISTING SUBMITTED VIA EFS-WEB

The content of the ASCII text file of the sequence listing named “UCLA266_ST25” which is 56 kb in size was created on Sep. 27, 2019, and electronically submitted via EFS-Web herewith the application, is incorporated herein by reference in its entirety.

### BACKGROUND

Lentiviral vectors are used as a gene transduction tool in both experimental and clinical settings that require long-term transgene expression. Their ability to integrate their transgenes into host chromosomes enables their transgenes to be expressed for a long period of time. Although integration of vectors into chromosomes enables long-term transgene expression, it can also cause insertional mutagenesis (destruction of host genes and their regulatory elements), so it is important to restrict the integration of the vectors only to the specific target cells.

Systemic administration of commonly used lentiviral vectors results in non-specific transduction of wide varieties of cells, due to the broad tropism of the envelope proteins used to pseudotype lentiviral vectors. Trapping of the vectors by the liver and spleen decreases the numbers of vectors available to reach other target organs, and transduction of the cells in the liver and spleen increases unnecessary insertional mutagenesis of these cells by vector integration.

There remains a need for efficient delivery of transgenes to target cells and tissues that avoids unnecessary transduction of non-target cells.

### SUMMARY

The vectors described herein escape this trapping in non-target organs, and specifically bind and transduce the target cell types. The lentiviral vectors pseudotyped with the novel envelope proteins described herein, such as E2 71 eMA and E2 71 mSAH, can be conjugated with biotinylated targeting ligands. The conjugated ligands mediate specific binding and transduction of the target cell types. This lentiviral transduction system can be used to selectively deliver transgenes into specific cell types in vivo, which increases the numbers of vectors reaching the targeted cells and tissues and decreases adverse effects in non-targeted cells and tissues. In addition, the vectors described herein specifically and efficiently transduce B cells without conjugation of targeting ligands. The B cell type most efficiently transduced in this manner is long-lived plasma cells. These results indicated that the specific transduction of this population by the targeting vector can be used for long-term transgene expression due to the ability of B-cells to induce tolerance to transgene products and long life span of long-lived plasma cells.

The invention provides, in one embodiment, a targeting construct comprising a nucleic acid sequence encoding a modified Sindbis virus envelope protein fused with a monomeric biotin-binding molecule. The modified Sindbis virus envelope protein comprises mutations in the E1, E2 and/or E3 proteins. Typically, the modified Sindbis virus envelope protein comprises mutant E2 and E3 proteins. In some embodiments, the mutant E2 protein lacks the sequence SLKQ. In some embodiments, the mutant E3 protein lacks the sequence RSKR.

Representative examples of the monomeric biotin-binding molecule include, but are not limited to, rhizavidin or a rhizavidin/streptavidin hybrid, including mutant forms of rhizavidin and streptavidin that retain high affinity binding to biotin in monomeric form. The monomeric biotin-binding molecule, or its equivalent, is capable of high affinity binding to biotin without forming multimers that could, for example, interfere with Sindbis virus envelope function. Illustrative embodiments of the targeting construct incorporating such a monomeric biotin-binding molecule include, but are not limited to, constructs comprising E2 71 eMA or E2 71 mSAH.

A targeting construct as described herein, or a retrovirus vector pseudotyped with the targeting construct, can further comprise a biotinylated targeting ligand conjugated with the biotin-binding molecule. Representative examples of a targeting ligand include, but are not limited to, an antibody or a receptor ligand, In a typical embodiment, the antibody is a monoclonal antibody. The targeting ligand is an agent that exhibits high affinity for target cells and little to no affinity for non-target cells. For example, the target cell could be a cancer cell, and the targeting ligand specifically binds a marker protein expressed by the target cancer cell. In another example, the target cell is of a particular tissue type, and the targeting ligand specifically binds a marker protein, surface antigen, receptor protein, that is expressed by cells of the target tissue.

Also provided is a pseudotyped retrovirus vector comprising a targeting construct as described herein. The vector optionally further comprises a heterologous gene. The vector, in some embodiments, comprises a retroviral nucleic acid genome, such as a lentiviral or an oncoretroviral genome. The nucleic acid genome can be RNA or DNA. The heterologous gene can be operably linked to a promoter or other expression control element, wherein the promoter is selected in accordance with the desired objective. Such promoters and control elements can be viral, heterologous, constitutive, inducible, etc. The heterologous gene is likewise selected in accordance with the desired objective, and can be a therapeutic gene, a corrective gene, a wild type gene, a cytotoxic gene, a marker gene, or other gene employed for therapeutic purposes, investigative purposes, and/or for detection.

The invention additionally provides a method of transducing a target cell with a heterologous gene. In one embodiment, the method comprises contacting the target cell with a pseudotyped retrovirus vector as described herein. In some embodiments, the contacting occurs in vitro or ex vivo. In other embodiments, the contacting occurs in vivo. The transduction of a target cell or cells can be for therapeutic or investigative purposes.

### BRIEF DESCRIPTION OF THE DRAWINGS

FIGS. 1A- B illustrate strategies to conjugate targeting antibodies to lentiviral vectors. (1A) Schematic representation of conjugating targeting antibodies to lentiviral vectors. Sindbis virus envelope proteins consist of two types of envelope proteins, E2, which mediates receptor binding, and E1, which mediates fusion between the cell membrane and viral envelope. The 2.2 pseudotype contains the ZZ peptide inserted into the E2 protein. The ZZ peptide binds the Fc region of antibodies. E2 71 AV, STAV, eMA, and mSAH have avidin, streptavidin, monomeric rhizavidin, and the monomeric streptavidin/rhizavidin hybrid, respectively, in E2. These molecules are known to bind biotin; therefore, the E2 71 AV, STAV, eMA, and mSAH pseudotypes are expected to be conjugated with biotinylated antibodies, (1B) The two integral membrane glycoproteins, E1 and E2, form a heterodimer and function as a unit. E3 and 6K work as signal sequence peptides for E2 and E1, respectively. 2.2 contains the ZZ peptide at aa 71 of E2, and 2.2 1L1 L replaces the ZZ peptide of 2.2 with flexible linkers encompassing restriction sites for cloning. E2 71 AV, STAV, eMA, and mSAH have core sequences of avidin, streptavidin, monomeric rhizavidin, and the monomeric rhizavidin/streptavidin hybrid between the flexible linkers at aa 71 of E2.

FIGS. 2A-2B show the expression of various envelope proteins on transfected cells and lentiviral vectors. (2A) 293T cells transfected with control vector (black line) or envelope protein expression vectors (gray line) were stained with anti-Sindbis virus antibody or biotinylated FITC and analyzed by flow cytometry. Flow cytometric profiles and mean fluorescence intensities (MFI) of staining with anti-Sindbis virus envelope protein antibody and biotinylated FITC are shown in bar graph. (2B) Western blotting analysis of lentiviral vectors pseudotyped with: \textcircled{1} 2.2 1L1L, \textcircled{2} E2 71 AV, \textcircled{3} E2 71 STAV, \textcircled{4} E2 71 eMA, \textcircled{5} E2 71 mSAH, or \textcircled{6}2.2. The same amounts of vectors (110 ng p24) were subjected to SDS-PAGE. After western blotting, the blotted membranes were stained with anti-Sindbis virus antibody, biotinylated HRP, or rabbit IgG-conjugated HRP.

FIGS. 3A-3B show targeted transduction with biotinylated antibodies. (3A) Jurkat and NIH3T3 cells were stained with biotinylated anti-hTfR1, mCD34, or isotype control antibodies, followed by staining with Alexa 488-conjugated streptavidin. The staining was analyzed by flow cytometry. (3B) Jurkat cells (1×105 cells) were infected with the same amount (1 ng p24/200 μl) of 2.2, 2.2 1L1L, E2 71 eMA, or E2 71 mSAH pseudotypes pre incubated with or without targeting ligands (triangle, no ligand; circle, anti-hTfR1 antibody; thick line, biotinylated anti-hTfR1 antibody; thin line with triangles, biotinylated isotype control antibody) at different concentrations. Transgene (EGFP) expression was analyzed by flow cytometry 3 days post-transduction. The averages and standard derivations of triplicate experiments are shown.

FIGS. 4A-4B. Properties of targeted transduction by the E2 71 eMA and E2 71 mSAH pseudotypes. (4A) 2.2, E2 71 eMA, and E2 71 mSAH were conjugated with biotinylated anti-hTfR1 antibody. The conjugated vectors were incubated with or without human serum (50%) for 1 hour at 37° C. and used to transduce Jurkat cells. EGFP transgene expression was analyzed by flow cytometry. The averages and standard deviations of the triplicate experiments are shown. (4B) E2 71 eMA and mSAH pseudotypes (100 ng p24/200 μl) were incubated with or without biotinylated anti-hTfR1 or mCD34 antibodies (200 pg). The vectors were then used to transduce mixtures of Jurkat (5×104) and CellTrace Violet-labeled NIH3T3 (2×104) cells. Three days post-transduction, the cells were stained with APC-conjugated anti-human CD47 antibody that specifically stains Jurkat cells. EGFP expression of Jurkat (APC+/CellTrace Violet−) and NIH3T3 (APC-/CellTrace Violet+) cells were analyzed by flow cytometry. The averages and standard derivations of triplicate experiments are shown.

FIGS. 5A-5F. Site-specific biotinylation of the anti-hTfR1 antibody. (5A) Schematic representation of conjugation of antibodies randomly biotinylated at lysine residues, the Fab fragment specifically biotinylated at C-terminal cysteines, and the biotinylated EGF. (5B) \textcircled{1} disulfide bonds between heavy chains of anti-hTfR1 antibody are reduced by 2-mercaptethanolamine; \textcircled{2} the reduced cysteines were biotinylated with maleimide biotin; and \textcircled{3} Fc regions of the biotinylated antibody are digested with pepsin. (5C) \textcircled{1} Biotinylated anti-hTfR1 antibody; and \textcircled{2} the Fab fragment of cysteine-biotinylated anti-hTfR1 antibody subjected to SOS-PAGE, followed by SyproRuby staining and western blotting, using HRP-conjugated streptavidin. (5D) Jurkat cells (1X105 cells) were infected with the same amount (1 ng p24/200 μl) of E2 71 eMA or E2 71 mSAH pseudotypes pre-incubated with or without biotinylated anti-hTfR1 antibody or biotinylated Fab fragment of anti-hTfR1 antibody at 200 ng/ml. Transgene (EGFP) expression was analyzed by flow cytometry 3 days post-transduction. The averages and standard derivations of triplicate experiments are shown. (5E) The titers (TU/40 μg p24/ml) of VSV-G pseudotype and the E2 71 eMA and mSAH pseudotypes with or without biotin α-hTfR1 Fab conjugation. The titers were calculated by triplicated transduction of Jurkat cells with EGFP-expressing vectors. The averages and standard derivations are shown. (5F) Flow cytometric profiles of Jurkat cells (1×105) transduced with VSV-G, E2 71 eMA or mSAH pseudotypes (1 ng p24/200 μl) conjugated with the Fab fragment of cysteine-biotinylated anti-hTfR1 antibody (10 ng) in the presence or absence of nevirapine (1 μM).

FIGS. 6A-6C. Targeted transduction by conjugation with biotinylated EGF. (6A) In upper panels, HeLa and Jurkat cells were stained with APC-conjugated anti-human EGFR (rightmost line), hTfR1 (center line), and its isotype control antibodies (leftmost line). In lower panels, Hela and Jurkat cells were incubated with biotinylated EGF (rightmost line) or buffer only (leftmost line), followed by incubation with APC-conjugated streptavidin. (6B) HeLa cells (5×104) were transduced with the same amount of wild-type Sindbis virus envelope protein and the E2 71 eMA and mSAH, or 2.2 pseudotype (100 ng p24/200 μl) with or without conjugation with various concentrations of biotinylated EGF. Jurkat cells (1×105) were transduced with the E2 71 eMA and mSAH pseudotypes (100 ng p24/200 μl) with or without conjugation with biotinylated EGF (0.2-200 ng/ml) or anti-hTfR1 antibody (100 ng/ml). EGFP transgene expression was analyzed 3 days post-transduction. The averages and standard derivations of the triplicate experiments are shown. (6C) HeLa cells (5×104) were incubated with anti-EGFR antibody (1 or 10 μg/ml) or its isotype control antibody (10 μg/ml), followed by transduction with the E2 71 eMA or mSAH pseudotypes (100 ng p24/200 μl) with or without conjugation with biotinylated EGF (20 ng/m1) or anti-hTfR1 antibody (200 ng/m1). EGFP transgene expression was analyzed 3 days post-transduction. The averages and standard derivations of the triplicate experiments are shown. Significance was calculated by comparing anti-hTfR1 antibody and EGF-mediated transduction without blocking antibody to those with blocking antibodies, using a two-sample two-sided unpaired student t-test (* *, p<0.01).

FIG. 7. Signaling elicited by EGF conjugated with the E2 71 eMA pseudotype. Phosphorylation of EGFR of Hela cells after 30 sec, 1 min, 5 min, 10 min, 30 min, 1 hour, or 2 hours of incubation with 200 μl of the E2 71 eMA or 2.2 pseudotype (100 ng p24/100 μl) and biotinylated EGF (2 ng/ml). The averages and standard derivations of the triplicate experiment are shown.

FIG. 8 illustrates significant transduction of the spleen of immunocompetent mice by the targeting lentiviral vector. The lentiviral vector, with Firefly luciferase as the transgene, was intravenously injected into immunocompetent mice (C57BL6) and transgene (luciferase) expression 5 days after injection is shown.

FIG. 9 demonstrates transduction of B-cells by intravenous administration of targeting vector based on flow cytometry. Vector harboring EGFP as its transgene was intravenously injected into immunocompetent mice (C57BL6) and transgene (EGFP) expression in the splenic cells was measured 5 days after injection. The splenic cells were isolated and stained with cell surface markers of immune cells. EGFP expression in each immune cells types was analyzed by flow cytometry. Results show that transgene (EGFP) is manly expressed in CD19+ cells

FIG. 10 shows different spleen cell subpopulations, illustrated schematically in upper panel, and percent of EGFP-transduced cells with markers of various B-cell subpopulations. Flow cytometry analysis showed that the cell type most efficiently transduced is long-lived plasma cells.

FIG. 11 is a schematic illustration of a lentiviral vector pseudotyped with modified Sindbis virus envelope protein binding to a B-cell receptor on the cell membrane of a B-cell.

### DETAILED DESCRIPTION

Described herein is a simple method of stable conjugation of lentiviral vectors with targeting ligands. Approaches for conjugating lentiviral vectors with targeting ligands are largely categorized as either covalent or non-covalent conjugation. The first involves expression of targeting ligands on the viral envelope by making fusion proteins of envelope proteins or membrane-anchoring proteins with targeting ligands. While conjugation by this method is stable, conjugation of each targeting ligand requires DNA cloning and validation of structures and expression levels. Additionally, the functions of fusion proteins must be retained for each targeting ligand, as fusion of targeting molecules sometimes affects the function of envelope proteins and/or targeting ligands.

The other method is to conjugate targeting molecules non-covalently to the vectors that have adaptor molecules on their surfaces. With this approach, once the function and expression levels of the adapt or molecule on the viral surface are validated, it is not necessary to clone expression plasmids for different types of target molecules and check their properties for every change of targeting ligands. In previous work, the ZZ peptide, which is an IgG Fc-binding peptide derived from protein A, was used as an adaptor molecule fused with the modified Sindbis virus envelope protein (Morizono et al., 2005; Pariente, et al., 2007; U.S. Pat. Nos. 8,449,875 and 9,163,248). The previous studies demonstrated that lentiviral vectors pseudotyped with the ZZ peptide-containing envelope protein can be conjugated with antibodies against various target molecules and specifically transduce cell types recognized by the conjugated antibodies. The conjugated antibodies, however, are detached in serum by competitive binding of serum antibodies to the ZZ domain.

To overcome this problem, a more stable conjugation method was developed using adaptor molecules that have higher affinity for their binding molecules. Although these approaches result in stable conjugation of targeting ligands, they require extensive engineering of targeting ligands to enable them to bind to adaptor molecules, which also requires validation of the structures and functions of the modified targeting molecules. Thus, for these targeting vectors to be widely and easily used by researchers, stable conjugation with minimally modified targeting ligands is preferable.

The vectors described herein, such as those pseudotyped with either E2 71 eMA or

E2 71 mSAH, can be stably conjugated with biotinylated antibodies and ligands by mixing the vectors with the ligands. Due to the simplicity of this method of stable ligand conjugation and ease of obtaining biotinylated ligands, this targeting system opens new avenues for the applications of lentiviral vectors in gene therapy and experimental research.

Surprisingly, these vectors described herein transduce B cells without use of targeting ligands. The subpopulation most efficiently transduced by the vector is long-lived plasma cells. The specific transduction of long-lived plasma cells by the targeting vector can be used for long-term transgene expression due to the ability of B cells to induce tolerance to transgene products and the long life span of long-lived plasma cells,

### Definitions

All scientific and technical terms used in this application have meanings commonly used in the art unless otherwise specified. As used in this application, the following words or phrases have the meanings specified.

As used herein, “Sindbis envelope,” refers to a viral envelope comprising the Sindbis E1, E2, and E3 proteins. The terms “Sindbis E1 protein,” “Sindbis E2 protein” and “Sindbis E3 protein” or a nucleic acid encoding “Sindbis E1protein,” “Sindbis E2 protein” and “Sindbis E3 protein” refer to nucleic acids and polypeptide polymorphic variants, alleles, mutants, and interspecies homologs that: (1) have a nucleotide sequence that has greater than about 60% nucleotide sequence identity, 65%, 70%, 75%, 80%, 85%, 90%, preferably 91%, 92%, 93%, 94%, 95%, 96%, 97%, 98% or 99% or greater nucleotide sequence identity, preferably over a region of at least about 25, 50, 100, 200, 500, 1000, or more nucleic acids, up to the full length sequence, to the nucleotide sequence of E1, E2, and/or E3; (2) bind to antibodies, e.g., polyclonal or monoclonal antibodies, raised against an immunogen comprising an amino acid sequence of an E1, E2, and/or E3 protein, and conservatively modified variants thereof: (3) specifically hybridize under stringent hybridization conditions to an anti-sense strand corresponding to a nucleic acid sequence of E1, E2, and/or E3 and conservatively modified variants thereof: (4) encode a protein having an amino acid sequence that has greater than about 60% nucleotide sequence identity, 65%, 70%, 75%, 80%, 85%, 90%, preferably 91%, 92%, 93%, 94%, 95%, 96%, 97%, 98% or 99% or greater nucleotide sequence identity, preferably over a region of at least about 25, 50, 100, 200, 500, 1000, or more amino acids, to a E1, E2, and/or E3 protein. The nucleic acids and proteins of the invention include both naturally occurring or recombinant molecules, as well as point mutations, including randomly generated point mutations and those generated by site-directed mutagenesis. E1, E2, and E3 are encoded by a polyprotein, the amino acid sequence of which is provided, e.g., by Accession No. VHWVB, VHWVB2, and P03316: the nucleic acid sequence is provided, e.g., by Accession No. SVU90536 and V01403 (see also Rice & Strauss, Proc. Nat'l Acad. Sci USA 78:2062-2066 (1981); and Strauss et al., Virology 133:92-110 (1984)). Other Togaviridae family envelopes, e.g., from the Alphavirus genus, e.g., Semliki Forest Virus, Ross River Virus, and equine encephalitis virus, can also be used to pseudotype the vectors of the invention. The envelope protein sequences for such Alphaviruses are known in the art.

“Pseudotype” refers to a virus particle, where the envelope or capsid includes heterologous viral proteins.

“Nucleic acid genome” refers to the genomic or nucleic acid component of a virus particle, which encodes the genome of the virus particle, including any proteins required for replication and/or integration of the genome, if required, and optionally a heterologous protein operably linked to a promoter, the promoter being either native to the protein or heterologous (viral or non-viral). The nucleic acid genome can be based on any virus, and have an RNA or DNA genome, either single stranded or double stranded. Preferably, the nucleic acid genome is from the family Retroviridae.

“Lentiviral vector” refers to viruses comprising nucleic acid genomes based on viruses of the Lentiviral genus of the family Retroviridae. Optionally, the vector encodes a heterologous gene.

“Retroviral vectors,” as used herein, refer to viruses based on viruses of the Retroviridae family. In their wild-type form, retroviral vectors typically contain a genomic nucleic acid. The pseudotyped, targeted retroviral vectors of the invention can optionally comprise a nucleic acid genome. The vectors of the invention can also comprise a heterologous gene.

“Targeting ligand” refers to a heterologous protein or fragment thereof that can be biotinylated and thereby conjugated to a pseudotyped virus particle of the invention. The targeting ligand binds to a protein on the cell surface of a selected cell type. Representative targeting moieties include antibodies and receptor ligands.

As used herein, a viral “envelope” protein, or “Env” protein refers to any polypeptide sequence that resides on the surface lipid bilayer of a retroviral virion whose function is to mediate the adsorption to and the penetration of host cells susceptible to infection. A retroviral envelope is formed by a cell-derived lipid bilayer into which proteins encoded by the env region of the viral genome are inserted. Envelope proteins are typically glycoproteins and usually comprise a transmembrane (TM) and a surface (SU) component linked together by disulfide bonds. Virus structure is described in detail in, for example, Coffin, et al., Retroviruses, 1997, Cold Spring Harbor Laboratory Press.

As used herein, a viral “capsid” refers to the principal structural protein of the virion core derived from the central region of the Gag polyprotein. The capsid protein in a mature viral particle forms a shell surrounding the ribonucleoprotein complex that contains the genomic nucleic acid. This shell, which includes additional proteins, is also referred to as a capsid. A capsid shell can exist as a component of a virion without surrounding a genomic nucleic acid.

As used herein, a “virion” refers to a retrovirus body, including the outer lipid bilayer which surrounds a capsid shell which in turn surrounds a genomic nucleic acid, when present. A virion of the invention, can, but need not, have a genomic nucleic acid.

As used herein, “mutated Sindbis envelope” refers to a point mutation, insertion, or deletion in the amino acid sequence of a wild-type Sindbis E1, E2, or E3 protein. The E1, E2, or E3 protein can have one or more mutations. In addition, combinations of mutations in E1, E2, and E3 are encompassed by the invention, e.g., mutations in E1and E2, or in E2 and E3, or E3 and E1, or E1, E2, and E3. Exemplary wild type sequences of E1, E2, and E3 proteins from Sindbis strains include Accession No, VHWVB, VHWVB2, and P03316.

The terms “identical” or percent “identity,” in the context of two or more nucleic acids or polypeptide sequences, refer to two or more sequences or subsequences that are the same or have a specified percentage of amino acid residues or nucleotides that are the same (i.e., about 60% identity, preferably 65%, 70%, 75%, 80%, 85%, 90%, 91%, 92%, 93%, 94%, 95%, 96%, 97%, 98%, 99%, or higher identity over a specified region, when compared and aligned for maximum correspondence over a comparison window or designated region) as measured using a BLAST or BLAST 2.0 sequence comparison algorithms with default parameters described below, or by manual alignment and visual inspection (see, e.g., NCBI web site http://www.ncbi.nlm.nih.gov/BLAST/or the like). Such sequences are then said to be “substantially identical.” This definition also refers to, or may be applied to, the compliment of a test sequence. The definition also includes sequences that have deletions and/or additions, as well as those that have substitutions. As described below, the preferred algorithms can account for gaps and the like. Preferably, identity exists over a region that is at least about 25 amino acids or nucleotides in length, or more preferably over a region that is 50-100 amino acids or nucleotides in length.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Preferably, default program parameters can be used, or alternative parameters can be designated. The sequence comparison algorithm then calculates the percent sequence identities for the test sequences relative to the reference sequence, based on the program parameters.

A “comparison window”, as used herein, includes reference to a segment of any one of the number of contiguous positions selected from the group consisting of from 20 to 600, usually about 50 to about 200, more usually about 100 to about 150 in which a sequence may be compared to a reference sequence of the same number of contiguous positions after the two sequences are optimally aligned. Methods of alignment of sequences for comparison are well-known in the art. Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Bioi. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by manual alignment and visual inspection (see, e.g., Current Protocols in Molecular Biology (Ausubel et al., eds. 1995 supplement)).

A preferred example of algorithm that is suitable for determining percent sequence identity and sequence similarity are the BLAST and BLAST 2.0 algorithms, which are described in Altschul et al., Nuc. Acids Res. 25:3389-3402 (1977) and Altschul et al., J. Mol. Bioi. 215:403-410 (1990), respectively. BLAST and BLAST 2.0 are used, with the parameters described herein, to determine percent sequence identity for the nucleic acids and proteins of the invention. Software for performing BLAST analyses is publicly available through the National Center for Biotechnology Information (http://www.ncbi.nlm.nih.gov/). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) of 10, M=S, N=−4 and a comparison of both strands. For amino acid sequences, the BLASTP program uses as defaults a wordlength of 3, and expectation (E) of 10, and the BLOSUM62 scoring matrix (see Henikoff & Henikoff, Proc. Nal. Acad. Sci. USA 89:10915 (1989)) alignments (B) of 50, expectation (E) of 10, M=S_{;} N=−4, and a comparison of both strands.

“Nucleic acid” refers to deoxyribonucleotides or ribonucleotides and polymers thereof in either single- or double-stranded form, and complements thereof. The term encompasses nucleic acids containing known nucleotide analogs or modified backbone residues or linkages, which are synthetic, naturally occurring, and non-naturally occurring, which have similar binding properties as the reference nucleic acid, and which are metabolized in a manner similar to the reference nucleotides. Examples of such analogs include, without limitation, phosphorothioates, phosphoramidates, methyl phosphonates, chiral-methyl phosphonates, 2-0-methyl ribonucleotides, peptide-nucleic acids (PNAs).

Unless otherwise indicated, a particular nucleic acid sequence also implicitly encompasses conservatively modified variants thereof (e.g., degenerate codon substitutions) and complementary sequences_{;} as well as the sequence explicitly indicated. Specifically, degenerate codon substitutions may be achieved by generating sequences in which the third position of one or more selected (or all) codons is substituted with mixed-base and/or deoxyinosinc residues (Batzer et al,, Nucleic Acid Res. 19:5081 (1991); Ohtsuka et al., J. Bioi. Chem, 260:2605-2608 (1985); Rossolini et al., Mol. Cell. Probes 8:91-98 (1994)). The term nucleic acid is used interchangeably with gene, eDNA, mRNA, oligonucleotide, and polynucleotide.

A particular nucleic acid sequence also implicitly encompasses “splice variants.” Similarly, a particular protein encoded by a nucleic acid implicitly encompasses any protein encoded by a splice variant of that nucleic acid. “Splice variants,” as the name suggests, are products of alternative splicing of a gene. After transcription, an initial nucleic acid transcript may be spliced such that different (alternate) nucleic acid splice products encode different polypeptides. Mechanisms for the production of splice variants vary, but include alternate splicing of exons. Alternate polypeptides derived from the same nucleic acid by read-through transcription are also encompassed by this definition. Any products of a splicing reaction, including recombinant forms of the splice products, are included in this definition. An example of potassium channel splice variants is discussed in Leicher, et al., J. Bid. Chem. 273(52):35095-35101 (1998).

The terms “polypeptide,” “peptide” and “protein” are used interchangeably herein to refer to a polymer of amino acid residues. The terms apply to amino acid polymers in which one or more amino acid residue is an artificial chemical mimetic of a corresponding naturally occurring amino acid, as well as to naturally occurring amino acid polymers and non-naturally occurring amino acid polymer.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, hydroxyproline, y-carboxyglutamate, and O-phosphoserine. Amino acid analogs refers to compounds that have the same basic chemical structure as a naturally occurring amino acid, i.e., an a carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions in a manner similar to a naturally occurring amino acid.

“Conservatively modified variants” applies to both amino acid and nucleic acid sequences. With respect to particular nucleic acid sequences, conservatively modified variants refers to those nucleic acids which encode identical or essentially identical amino acid sequences, or where the nucleic acid does not encode an amino acid sequence, to essentially identical sequences. Because of the degeneracy of the genetic code, a large number of functionally identical nucleic acids encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein which encodes a polypeptide also describes every possible silent variation of the nucleic acid. One of skill will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid which encodes a polypeptide is implicit in each described sequence with respect to the expression product, but not with respect to actual probe sequences.

As to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles of the invention.

The following eight groups each contain amino acids that are conservative substitutions for one another: 1) Alanine (A), Glycine (G); 2) Aspartic acid (0), Glutamic acid (E); 3) Asparagine (N), Glutamine (Q); 4) Arginine (R), Lysine (K); 5) Isoleucine (1), Leucine (L), Methionine (M), Valine (V); 6) Phenylalanine (F), Tyrosine (Y), Tryptophan (W); 7) Serine (S), Threonine (T); and 8) Cysteine (C), Methionine (M) (see, e.g., Creighton, Proteins (1984)).

A “label” or a “detectable moiety” is a composition detectable by spectroscopic, photochemical, biochemical, immunochemical, chemical, or other physical means. For example, useful labels include 32P, fluorescent dyes, electron-dense reagents, enzymes (e.g., as commonly used in an ELISA), biotin, digoxigenin, or haptens and proteins which can be made detectable, e.g., by incorporating a radiolabel into the peptide or used to detect antibodies specifically reactive with the peptide.

The term “recombinant” when used with reference, e.g., to a cell, or nucleic acid, protein, or vector, indicates that the cell, nucleic acid, protein or vector, has been modified by the introduction of a heterologous nucleic acid or protein or the alteration of a native nucleic acid or protein, or that the cell is derived from a cell so modified. Thus, for example, recombinant cells express genes that are not found within the native (non-recombinant) form of the cell or express native genes that are otherwise abnormally expressed, under expressed or not expressed at all.

The term “heterologous” when used with reference to portions of a nucleic acid indicates that the nucleic acid comprises two or more subsequences that are not found in the same relationship to each other in nature. For instance, the nucleic acid is typically recombinantly produced, having two or more sequences from unrelated genes arranged to make a new functional nucleic acid, e.g. a promoter from one source and a coding region from another source. Similarly, a heterologous protein indicates that the protein comprises two or more subsequences that are not found in the same relationship to each other in nature (e.g., a fusion protein).

The phrase “stringent hybridization conditions” refers to conditions under which a probe will hybridize to its target subsequence, typically in a complex mixture of nucleic acids, but to no other sequences. Stringent conditions are sequence-dependent and will be different in different circumstances. Longer sequences hybridize specifically at higher temperatures. An extensive guide to the hybridization of nucleic acids is found in Tijssen, Techniques in Biochemistry and Molecular Biology—Hybridization with Nucleic Probes, “Overview of principles of hybridization and the strategy of nucleic acid assays” (1993). Generally, stringent conditions are selected to be about 5-10° C. lower than the thermal melting point (Tm) for the specific sequence at a defined ionic strength pH. The Tm is the temperature (under defined ionic strength, pH, and nucleic concentration) at which 50% of the probes complementary to the target hybridize to the target sequence at equilibrium (as the target sequences are present in excess, at Tm, 50% of the probes are occupied at equilibrium). Stringent conditions may also be achieved with the addition of destabilizing agents such as formamide. For selective or specific hybridization, a positive signal is at least two times background, preferably 10 times background hybridization. Exemplary stringent hybridization conditions can be as following: 50% formamide, 5×SSe, and 1% SDS, incubating at 42° C., or, 5×SSC, 1% SDS, incubating at 65° e, with wash in 0.2×SSC, and 0.1% SDS at 65° C.

Nucleic acids that do not hybridize to each other under stringent conditions are still substantially identical if the polypeptides which they encode are substantially identical. This occurs, for example, when a copy of a nucleic acid is created using the maximum codon degeneracy permitted by the genetic code, In such cases, the nucleic acids typically hybridize under moderately stringent hybridization conditions. Exemplary “moderately stringent hybridization conditions” include a hybridization in a buffer of 40% formamide, 1 M NaCI, 1% SDS at 37° C. and a wash in 1×SSC at 45° C. A positive hybridization is at least twice background. Those of ordinary skill will readily recognize that alternative hybridization and wash conditions can be utilized to provide conditions of similar stringency. Additional guidelines for determining hybridization parameters are provided in numerous reference, e.g., and Current Protocols in Molecular Biology, ed. Ausubel, et al.

For PCR, a temperature of about 36° C. is typical for low stringency amplification, although annealing temperatures may vary between about 32° C. and 48° C. depending on primer length. For high stringency PCR amplification, a temperature of about 62° C. is typical, although high stringency annealing temperatures can range from about 50° C. to about 65° C., depending on the primer length and specificity. Typical cycle conditions for both high and low stringency amplifications include a denaturation phase of 90° C.-95° C. for 30 sec-2 min., an annealing phase lasting 30 sec.-2 minₐ and an extension phase of about 72° C. for 1-2 min. Protocols and guidelines for low and high stringency amplification reactions are provided, e.g., in Innis et al. (1990) PCR Protocols, A Guide to Methods and Applications, Academic Press, Inc. N.Y.).

“Antibody” refers to a polypeptide comprising a framework region from an immunoglobulin gene or fragments thereof that specifically binds and recognizes an antigen. The recognized immunoglobulin genes include the kappa, lambda, alpha, gamma, delta, epsilon, and mu constant region genes, as well as the myriad immunoglobulin variable region genes. Light chains are classified as either kappa or lambda. Heavy chains are classified as gamma, mu, alpha, delta, or epsilon, which in turn define the immunoglobulin classes, IgG. IgM, IgA, IgD and IgE, respectively. Typically, the antigen-binding region of an antibody will be most critical in specificity and affinity of binding. An exemplary immunoglobulin (antibody) structural unit comprises a tetramer. Each tetramer is composed of two identical pairs of polypeptide chains, each pair having one “light” (about 25 kD) and one “heavy” chain (about 50-70 kD). The N-terminus of each chain defines a variable region of about 100 to 110 or more amino acids primarily responsible for antigen recognition. The terms variable light chain (VL) and variable heavy chain (VH) refer to these light and heavy chains respectively.

A “chimeric antibody” is an antibody molecule in which (a) the constant region, or a portion thereof, is altered, replaced or exchanged so that the antigen binding site (variable region) is linked to a constant region of a different or altered class. effector function and/or species, or an entirely different molecule which confers new properties to the chimeric antibody, e.g., an enzyme, toxin, hormone, growth factor, drug, etc.; or (b) the variable region, or a portion thereof, is altered, replaced or exchanged with a variable region having a different or altered antigen specificity.

As used herein, “specifically (or selectively) binds” to an antibody or “specifically (or selectively) immunoreactive with,” when referring to a protein or peptide, refers to a binding reaction that is determinative of the presence of the protein, often in a heterogeneous population of proteins and other biologics. Thus, under designated immunoassay conditions, the specified antibodies bind to a particular protein at least two times the background and more typically more than 10 to 100 times background. Specific binding to an antibody under such conditions requires an antibody that is selected for its specificity for a particular protein.

As used herein, “therapeutically effective dose” is a dose that produces effects for which it is administered. The exact dose will depend on the purpose of the treatment, and will be ascertainable by one skilled in the art using known techniques (see, e.g. Lieberman, Pharmaceutical Dosage Forms (vols. 1-3, 1992); Lloyd, The Art, Science and Technology of Pharmaceutical Compounding (1999); and Pickar, Dosage Calculations (1999)). As used herein, “pharmaceutically acceptable carrier” or “excipient” includes any material which, when combined with an active ingredient, allows the ingredient to retain biological activity and is non-reactive with the subject's immune system. Examples include, but are not limited to, any of the standard pharmaceutical carriers such as a phosphate buffered saline solution, water, emulsions such as oiliwater emulsion, and various types of wetting agents. Preferred diluents for aerosol or parenteral administration are phosphate buffered saline or normal (0.9%) saline.

Compositions comprising such carriers are formulated by well-known conventional methods (see, for example, Remington's Pharmaceutical Sciences, 18th edition, A. Gennaro, ed., Mack Publishing Co., Easton, Pa., 1990).

As used herein, the term “subject” includes any human or non-human animal. The term “non-human animal” includes all vertebrates, e.g., mammals and non-mammals, such as non-human primates, horses, sheep, dogs, cows, pigs, chickens, and other veterinary subjects. In a typical embodiment, the subject is a human.

As used herein, “a” or “an” means at least one, unless clearly indicated otherwise.

### Constructs and Vectors

The invention provides, in one embodiment, a targeting construct comprising a nucleic acid sequence encoding a modified Sindbis virus envelope protein fused with a monomeric biotin-binding molecule. The modified Sindbis virus envelope protein comprises mutations in the E1, E2 and/or E3 proteins, In some embodiments, the modified Sindbis virus envelope protein comprises mutant E2 and E3 proteins. In some embodiments, the mutant E2 protein lacks the sequence SLKQ. In some embodiments, the mutant E3 protein lacks the sequence RSKR.

Representative examples of the monomeric biotin-binding molecule include, but are not limited to, rhizavidin or a rhizavidin/streptavidin hybrid, including mutant forms of rhizavidin and streptavidin that retain high affinity binding to biotin in monomeric form. The monomeric biotin-binding molecule, or its equivalent, is capable of high affinity binding to biotin without forming multimers that could, for example, interfere with Sindbis virus envelope function. Illustrative embodiments of the targeting construct incorporating such a monomeric biotin-binding molecule include, but are not limited to, constructs comprising E2 71 eMA (SEQ ID NO: 5; encoding SEQ ID NO: 6) or E2 71 mSAH (SEQ ID NO: 7; encoding SEQ ID NO: 8).

In some embodiments, a targeting construct as described herein, or a retrovirus vector pseudotyped with the targeting construct, further comprises a biotinylated targeting ligand conjugated with the biotin-binding molecule. Representative examples of a targeting ligand include, but are not limited to, an antibody or a receptor ligand. In a typical embodiment, the antibody is a monoclonal antibody. The targeting ligand is an agent that exhibits high affinity for target cells and little to no affinity for non-target cells. For example, the target cell could be a cancer cell, and the targeting ligand specifically binds a marker protein expressed by the target cancer cell. In another example, the target cell is of a particular tissue type, and the targeting ligand specifically binds a marker protein, surface antigen, receptor protein, that is expressed by cells of the target tissue.

Also provided is a pseudotyped retrovirus vector comprising a targeting construct as described herein. The vector optionally further comprises a heterologous gene. The vector, in some embodiments, comprises a retroviral nucleic acid genome, such as a lentiviral or an oncoretroviral genome. The nucleic acid genome can be RNA or DNA. The heterologous gene can be operably linked to a promoter or other expression control element, wherein the promoter is selected in accordance with the desired objective. Such promoters and control elements can be viral, heterologous, constitutive, inducible, etc. The heterologous gene is likewise selected in accordance with the desired objective, and can be a therapeutic gene, a corrective gene, a wild type gene, a cytotoxic gene, a marker gene, or other gene employed for therapeutic purposes, investigative purposes, and/or for detection.

### Methods

The invention additionally provides a method of transducing a target cell with a heterologous gene. In one embodiment, the method comprises contacting the target cell with a pseudotyped retrovirus vector as described herein. In some embodiments, the contacting occurs in vitro or ex vivo. In other embodiments, the contacting occurs in vivo. The transduction of a target cell or cells can be for therapeutic or investigative purposes.

In some embodiments, the target cell is a B cell. In some embodiments, the B cell is a long-lived plasma cell. In some embodiments, the target cell is a T cell.

Representative transgenes include, but are not limited to, a gene encoding a chimeric antigen receptor. In one representative example, one can transduce T-cells with chimeric antigen receptor transgene, providing a simple and less expensive means of ex vivo transduction-based CAR-T cell therapy. In another representative example, one can transduce B-cells, especially long-lived plasma cells, with one or more therapeutic antibody transgenes. Transduction of long-lived plasma cells results in long-term expression of therapeutic genes, providing a drastic improvement over antibody-based therapies that require multiple administration of recombinant antibodies.

To transduce T-cells, the ligands that specifically bind and activate T-cells can be conjugated with a targeting vector as described herein. Such ligands can include, but are not limited to, antibodies directed against CD3 and Interlukin-7.

To transduce B-cells, ligands that specifically bind and activate B-cells can be conjugated with a targeting vector as described herein. Such ligands can include, but are not limited to, protein L, sCD40 ligands, and antigens of B-cell receptors.

### EXAMPLES

The following examples are presented to illustrate the present invention and to assist one of ordinary skill in making and using the same. The examples are not intended in any way to otherwise limit the scope of the invention.

### Example 1:

### Versatile Targeting System for Lentiviral Vectors Involving Biotinylated Targeting Molecules

Conjugating certain types of lentiviral vectors with targeting ligands can redirect the vectors to specifically transduce desired cell types. However, extensive genetic and/or biochemical manipulations are required for conjugation, which hinders applications for targeting lentiviral vectors for broader research fields. This Example describes envelope proteins fused with biotin-binding molecules to conjugate the pseudotyped vectors with biotinylated targeting molecules by simply mixing them. The envelope proteins fused with the monomeric, but not tetrameric, biotin-binding molecules can pseudotype lentiviral vectors and be conjugated with biotinylated targeting ligands. The conjugation is stable enough to redirect lentiviral transduction in the presence of serum, indicating their potential in in viva When a signaling molecule is conjugated with the vector, the conjugation facilitates transduction and signaling in a receptor-specific manner. This simple method of ligand conjugation and ease of obtaining various types of biotinylated ligands will make targeted lentiviral transduction easily applicable to broad fields of research.

When systemically administered, lentiviral vectors pseudotyped with commonly used envelope proteins such as vesicular stornatitis virus glycoprotein (VSV-G) are trapped by the liver and/or spleen and transduce cells in these organs, which decreases the number of vector particles available to reach the target organs (Brown et al., 2006) (Morizono et al., 2005). To efficiently deliver transgenes to target cells and tissues and avoid unnecessary transduction of non-target cells in the liver and spleen, the vectors need to escape trapping and have the ability to specifically bind and transduce the desired cell types. Such vectors are called “targeting vectors” (Kasahara, Dozy, and Kan, 1994).

Because envelope proteins mediate binding of the pseudotypes to target cells, targeting lentiviral vectors are developed by changing the binding specificity of pseudotyping envelope proteins. This requires both eliminating their original tropisms and conferring binding affinities specific to the molecules expressed on target cells(Morizono and Chen, 2005).

The original tropisms of pseudotyping envelope proteins can usually be eliminated by mutating their receptor-binding regions and they are then used as backbones to conjugate the specific targeting ligands(Morizono and Chen, 2011; Nakamura et al,, 2005). The tropisms of lentiviral vectors has been modified by pseudotyping the vectors with modified Sindbis virus envelope proteins(Morizono et al., 2001; Morizono et al., 2010; Morizono et al., 2009a; Morizono et al., 2009b; Morizono et al., 2005). The Sindbis virus has two envelope proteins, E2, which mediates binding, and E1, which mediates fusion(Fields, Knipe, and Howley, 2013; Ohno et al., 1997). Several receptor-binding regions of E2 were mutated to eliminate its original tropism (Dubuisson and Rice, 1993; Klimstra, Heidner, and Johnston, 1999; Morizono et al., 2005). This mutated Sindbis envelope protein that lacks its natural tropism provides an ideal basis to develop a targeting lentiviral vector by conjugation with targeting ligands(Ahani et al., 2016; Aires da Silva et al., 2005; Bergman et al., 2004; Kasaraneni et al., 2017; Kasaraneni et al., 2018; Yang et al., 2006).

Approaches for conjugating targeting ligands are largely categorized as either covalent or non-covalent conjugation. The first involves expression of targeting ligands on the viral envelope by making fusion proteins of envelope proteins or membrane-anchoring proteins with targeting ligands. While conjugation by this method is stable, conjugation of each targeting ligand requires DNA cloning and validation of structures and expression levels(Bender et al., 2016; Funke et al., 2008; Kasahara, Dozy, and Kan, 1994; Munch et al., 2011; Nakamura et al., 2005; Sandrin, Russell, and Cosset, 2003; Somia, Zoppe, and Verma, 1995). Additionally, the functions of fusion proteins must be retained for each targeting ligand, as fusion of targeting molecules sometimes affects the functions of envelope proteins and/or targeting ligands(Fielding et al., 1998). For example, fusion of murine leukemia virus envelope proteins with targeting ligands results in loss of the fusion activity of the envelope protein, which is indispensable for transduction(Zhao et al,, 1999). The other method is to conjugate targeting molecules non-covalently to the vectors that have adaptor molecules on their surfaces. In this approach, once the function and expression levels of the adaptor molecule on the viral surface are validated, it is not necessary to clone expression plasmids for different types of target molecules and confirm those properties every time the targeting ligands are changed.

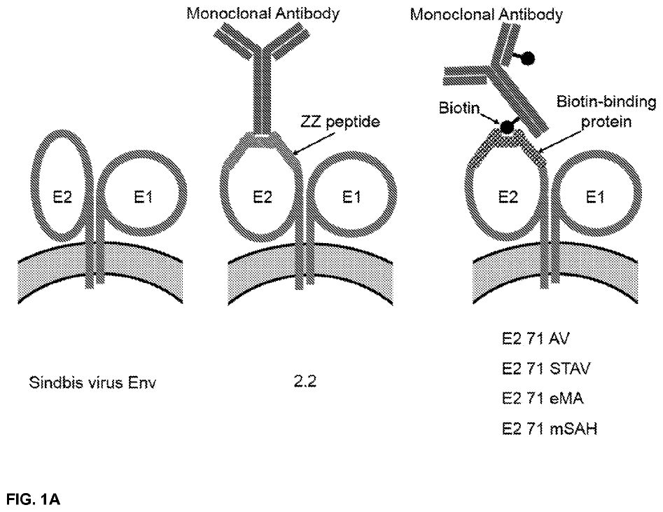

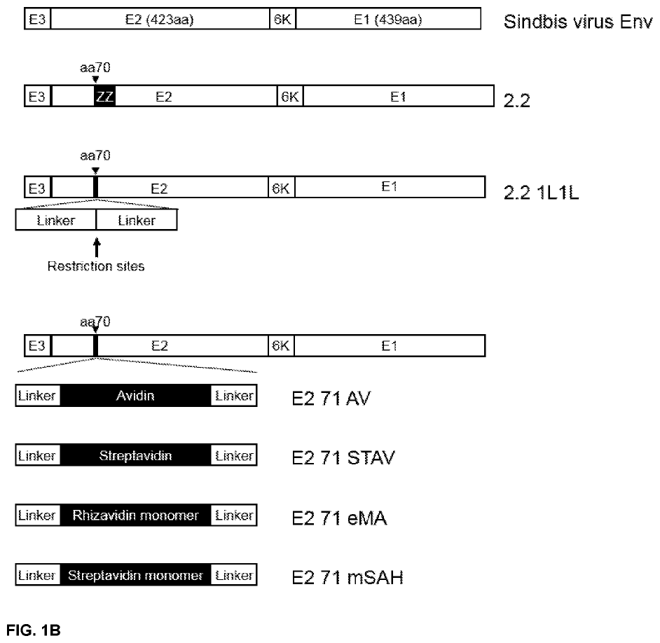

The ZZ peptide, IgG Fc-binding peptide derived from protein A, was previously used as an adaptor molecule fused with the mutated Sindbis virus envelope protein (2.2, FIG. 1A and 1B; nucleotide sequence shown in SEQ ID NO: 3; amino acid sequence shown in SEQ ID NO: 4) to non-covalently conjugate targeting antibodies to lentiviral vectors. Lentiviral vectors pseudotyped with 2.2 can be easily conjugated with antibodies against various target molecules, including CD4, Transferrin receptor 1 (TfR1), PSCA, CD19, CD20, DC-SIGN, CD34, and P-glycoprotein, by simply mixing the vectors with antibodies (Liang et al., 2009a; Liang et al., 2009b; Morizono et al., 2001; Morizono et al., 2010; Morizono et al., 2006; Morizono et al., 2005; Pariente et al., 2007). The vectors specifically transduce cell types recognized by the conjugated antibodies. Due to the ease of conjugating antibodies, this targeting lentiviral vector system has been successfully used (Anderson et al., 2009; Bergman et al., 2004; Cao et al., 2016; Lafitte et al., 2012; Wu et al., 2012; Zhang et al., 2011; Zhang et al., 2009; Zhang and Roth, 2010). However, the conjugated antibodies are detached by competitive binding of serum antibodies to the ZZ domain when serum immunoglobulin is present(Morizono et al., 2010).

Therefore, the current applications of this targeting lentiviral vector are limited to in vitro settings and an immunodeficient mouse model that does not have serum immunoglobulin(Liang et al., 2009a; Liang et al., 2009b; Morizono et al., 2005; Pariente et al., 2008; Pariente et al., 2007). More stable conjugation methods using adaptor molecules that have higher affinity for their binding molecules need to be developed to overcome this problem.

Avidin and streptavidin are known to bind biotin at exceptionally high affinities. The dissociation constant (Kd) of binding between these two molecules is 10-15, which is 107-8 less than the Kd of the binding between the ZZ domain and the Fc region of antibodies (Laitinen et al., 2006). Therefore, molecules fused with them can be conjugated with biotinylated targeting ligands.

One group conjugated biotinylated antibodies on lentiviral vectors pseudotyped with both membrane-anchored avidin or streptavidin (for conjugation with biotinylated targeting ligands) and wild-type baculovirus envelope protein (for subsequent fusion) (Kaikkonen et al., 2009) (Huhtala et al., 2014) . Targeting antibodies were stably conjugated in both in vitro and in vivo settings, demonstrating the usefulness of avidin/biotin interactions for conjugation of virus with ligands. However, the targeting specificity of this vector is poor because this pseudotype still retains the broad tropism of pseudotyping baculovirus envelope protein. These studies indicated that the original receptor-binding regions of pseudotyping envelope proteins should be abolished for highly specific targeting lentiviral vectors, even when strong avidin-biotin interactions are used for ligand conjugation.

In the present Example, the strong binding between biotin-binding molecules derived from streptavidin and rhizavidin with biotin and a mutated Sindbis virus envelope backbone was used to redirect the pseudotyped lentiviral vectors by stable conjugation with biotinylated targeting ligands.

### Materials and Methods

Plasmids, antibodies, proteins and chemicals,

Expression vectors of wild-type Sindbis virus envelope protein 2.2 and 2.2 1L1L were described previously (Morizono et al., 2001; Morizono et al., 2009a; Pariente et al., 2007). Expression vectors of E2 71 AV, STAY, eMA, and mSAH were constructed by inserting the core sequences of Avidin, streptavidin, monomeric rhizavidin, and monomeric streptavidin/rhizavidin hybrids, respectively, between flexible linkers at amino acid (aa) 71 of E2. Anti-hTfR1 and EGFR antibodies were purchased from Bio X Cell (West Lebanon, N.H). Biotin and APC-conjugated anti-hTfR1 antibody, Alexa 488 and HRP-conjugated streptavidin, goat Alexa 488 and HRP-conjugated anti-mouse IgG, Alexa 488-conjugated goat anti-rabbit IgG, and biotin-conjugated EGF were purchased from ThermoFisher Scientific (Canoga Park, Calif.). Biotin-conjugated anti-mCD34 antibody, APC-conjugated anti-human EGFR antibody, and APC-conjugated anti-human CD47 antibody were purchased from Biolegend (San Diego, Calif.). Biotinylated FITC was purchased from Sigma-Aldrich (St. Louis, Mo.).

Cells and Viruses.

293T cells were cultured in DMEM (ThermoFisher Scientific) containing 10% dialyzed FCS (Sigma-Aldrich) and antibiotics. Jurkat, NIH3T3_{;} and HeLa cells were cultured in IMDM (ThermoFisher Scientific) containing 10% BaCl2-precipitated FCS. All lentiviral vectors were produced in 293T cells, using TransIT LT1 (Mirus Bio, Madison, Wis.). Briefly, 293T cells (1.4×107) were transfected with one type of protein expression vector (6-7 μg), packaging plasmid ps PAX2, (6-7 pg), and either lentiviral vector or cppt2e (6-7 μg). Two days post-transfection, the supernatant was subjected to ultracentrifugation (20,000 rpm, 4° C., 2 hours) by SW32 rotor (Beckman-Coulter, Brea, Calif.), using PBS containing 25% sucrose and 1 mM EGTA. The pellet containing the virus was resuspended in Hanks buffered saline (100-fold concentration).

Western Blotting.

The amounts of viral vectors were normalized to the amount of HIV p24 (1 mg/ml) and mixed with LDS sample buffer (ThermoFisher Scientific) with 2-mercaptoethanol. Each sample (20 pl) was subjected to electrophoresis through an SOS 12% polyacrylamide gel (ThermoFisher Scientific). Immunoblot analyses of envelope proteins were performed with: 1) rabbit anti-Sindbis virus polyclonal antibody and horseradish peroxidase (HRP)-conjugated goat anti-rabbit polyclonal antibody (ThermoFisher Scientific); 2) HRP-conjugated rabbit goat anti-goat immunoglobulin antibody (ThermoFisher Scientific); and 3) biotinylated HRP (ThermoFisher Scientific). The protein bands were visualized by ECL plus substrate (ThermoFisher Scientific) and ChemiDoc MP Imaging System (Bio-Rad, Hercules, Calif.).

Site-specific biotinylation of OKT9.

The disulfide bonds of the hinge region of OKT9 were reduced by incubation with 50 mM β-mercaptoethylamine (ThermoFisher Scientific) for 90 min at 37° C., and the reduced OKT9 was biotinylated with EZ-Link Maleimide-PEG2-Biotin (ThermoFisher Scientific) according to the manufacturer's protocol. The Fab fragment of biotinylated OKT9 was generated with the F(ab′)2 preparation kit (ThermoFisher Scientific) according to the manufacturer's protocol. Fragmentation of biotinylated OKT9 and biotinylation of the heavy and light chains were analyzed by SyproRuby staining (ThermoFisher Scientific) and western blotting, using HRP-conjugated streptavidin, respectively, following SDS-PAGE, using a 12% polyacrylamide gel in a reduced condition.

Flow cytometric analysis of the modified Sindbis virus envelope proteins hTfR1, mCD34, and human EGFR,

293T cells were transfected with expression vectors of wild-type Sindbis virus envelope protein, 2.2 1L1L, E2 71 AV, E2 71 STAV, E2 71 eMA, or E2 71 mSAH, using TransIT LT1. Two days post-transfection, expression of the envelope proteins was analyzed by staining the transfected cells with rabbit anti-Sindbis virus antibody, followed by staining with Alexa 488-conjugated goat anti-rabbit IgG antibody (ThermoFisher Scientific). The binding of biotin was analyzed by staining the transfected cells with biotinylated FITC. Expression of hTfR1 and mCD34 on Jurkat and NIH3T3 cells was analyzed by staining cells with either anti-hTfR1, mCD34, or isotype control antibodies, followed by staining with Alexa 488-conjugated streptavidin (ThermoFisher Scientific). Expression of hTfR1 and EGFR on HeLa and Jurkat cells was analyzed by staining the cells with ARC-conjugated anti-hTfR1, EGFR, or its isotype control antibody. Binding of EGFR to HeLa and Jurkat cells was analyzed by staining the cells with biotinylated EGF, followed by staining with ARC-conjugated streptavidin (Biolegend). Flow cytometric data were acquired by FACScan (BD) upgraded with a red laser (Cytek, Fremont, Calif.) and analyzed by FCSExpress 5 (De Novo Software, Los Angeles, Calif.).

Transduction of Jurkat and NIH3T3 cells, 2.2, and 2.2 1L1L.

E2 71 eMA and E2 71 mSAH (2 ng p24/ml) were conjugated with different concentrations of anti-hTfR1, biotinylated anti-hTfR1, biotinylated isotype control antibodies, or the biotinylated Fab fragment of anti-hTfR1 at room temperature for 30 min. Cells (1×105) were transduced with 200 μl of virus at 37° C. for 2 hours. After transduction, cells were cultured for 3 days. EGFP expression was analyzed by flow cytometry. NIH3T3 cells were labeled with CellTrace Violet (ThermoFisher Scientific). The labeled NIH3T3 (2×104) and Jurkat cells (5×104) were infected with 200 pL of E2 71 eMA or mSAH (100 ng p24/ml) with or without conjugation of 500 ng/ml biotinylated anti-hTFR1 or mCD34 antibodies. After incubation with the vectors for 2 hours, cells were cultured in medium for 3 days. The cells were then harvested and stained with ARC-conjugated anti-human CD47 antibody. Flow cytometric data were acquired by BDFortessa. EGFP expression of Jurkat cell populations (ARC-positive/CellTraceViolet-negative) and NI H3T3 cell populations (APC-negative/CellTraceViolet-positive) were analyzed by FCSExpress 5.

Inhibition of Jurkat transduction by human serum and Nevirapine.

The E2 71 eMA and mSAH pseudotypes (1 μg p24/m1) were conjugated with biotinylated anti-hTfR1 antibody (1 μg/m1) and incubated with or without 50% human AB serum (Sigma-Aldrich) for 1 hour at 37° C. The pseudotypes were diluted with PBS to 100 ng/ml p24/ml. Jurkat cells were incubated with 200 μL of the vectors, and EGFP expression was analyzed by flow cytometry 3 days post-transduction. For inhibition of reverse transcription, Jurkat cells were incubated with 20 nM Nevirapine (NIH AIDS Reagent Program) for 1 hour prior to transduction. The cells were then transduced with VSV-G, E2 71 eMA or mSAH pseudotype with or without conjugation of the biotinylated Fab fragment of anti-hTfR1 in the presence or absence of 20 nM Nevirapine. The cells were cultured for 3 days post-transduction in the absence or presence of 20 nM Nevirapine. EGFP expression was analyzed by flow cytometry 3 days post- transduction.

Transduction by EGF conjugation.

The E2 71 eMA, mSAH, and 2.2 pseudotypes (100 ng p24/mi) were incubated with different concentrations of biotinylated EGF for 30 min at room temperature. HeLa and Jurkat cells (1×105) were incubated with 200 μl of wild-type Sindbis virus envelope, 2.2, E2 71 eMA, or mSAH pseudotype with or without biotinylated EGF conjugation. Three days post-transduction, EGFP expression was analyzed by flow cytometry. To confirm the roles of EGFR in EGF-mediated transduction, HeLa cells were incubated with anti-EGFR (Bio X cell) or its isotype control (Biolegend) antibodies at 1 or 10 μg/ml, followed by transduction with E2 71 eMA or mSAH pseudotype (100 ng p24/ml) conjugated with biotinylated EGF (20 ng/ml) or anti-hTfR1 antibody (200 ng/ml) in the absence or presence of the blocking antibodies. EGFP expression was analyzed by flow cytometry 3 days post- transduction.

Quantification of EGFR phosphorylation.

HeLa cells were incubated with 2.2 or E2 71 eMA pseudotype (100 ng p24)/ml, then incubated with EGF-biotin (2 ng/ml) for 30 sec to 2 hours. Cells were promptly lysed after indicated incubation times in 10 mM Tris-HCI pH 8.0, 1 mM EDTA, 1% Triton-X 100, 0.1% Na deoxycholate, 0.1% SDS, and 140 mM NaCl, with protease and phosphatase inhibitor supplemented before use (Boston Bioproducts, Ashland, Ma.). Protein concentrations were quantitated with a bicinchoninic acid assay. A magnetic bead-based ELISA assay was used for phosphorylation measurement. Lysates were incubated with EGFR antibody-coupled magnetic beads (Bio-Rad Laboratories) overnight at 4 [symbol] C, then the beads were washed with 0.1% (v/v) Tween-20 in TBS. Phospho-tyrosine biotinylated antibody (R&D Systems) and streptavidin-phycoerythrin (Bio-Rad Laboratories) were incubated for 60 and 15 min, respectively, at room temperature. Signaling was quantified using a MagPix Luminex reader (Bio-Rad Laboratories), then normalized to protein concentration.

### Results

Designing biotin-binding envelope proteins.

The backbone construct, 2.2 1L1L, is derived from wild-type Sindbis virus with multiple mutations at the original receptor-binding regions (FIG. 1B) (Morizono et al., 2006; Morizono et al., 2005; Pariente et al., 2007). 2.2 1L1L contains restriction sites for inserting an adaptor molecule(s) between aa 71 and 74 of E2, which has flexible linkers before and after the insertion sites (Morizono et al., 2009a; Morizono et al., 2009b). At the insertion site, 2.2 contains the ZZ peptide (FIG. 1A and B). Since the conjugation of the ZZ domain with antibodies is unstable in serum, the development of strategies to stably conjugate targeting ligands to the targeting vectors was attempted. Because the binding of biotin and avidin/streptavidin is known to be stable in various in vivo and in vitro settings due to its extremely high affinity (Bayer and Wilchek, 1990), avidin or streptavidin was inserted into 2.2 1L1 L, designated E2 71 AV and E2 71 STAV, respectively (FIG. 1A and B). Because the Sindbis virus envelope proteins form trimers (Li et al., 2010), whereas avidin and streptavidin form tetramers (Bayer and Wilchek, 1990), it is possible that fusion of the Sindbis envelope proteins with avidin/streptavidin impairs proper multimerization and structures on both sides. Thus, monomeric biotin-binding molecules eMA and mSAH were inserted instead of avidin and streptavidin into 2.21L1L, designated E2 71 eMA and E2 71 mSAH, respectively (FIG. 1A and B). eMA is a monomeric form mutant of rhizavidin, which is a dimeric biotin-binding molecule isolated from Rhizobium etli (Helppolainen et al., 2007; Lee et al., 2016). mSAH is a monomeric streptavidin developed by combining amino acid sequences of streptavidin with rhizavidin (Demonte et al., 2013). It was reported that the Kd of eMA and biotin was 3.1×10-11, and dissociation of eMA bound to biotin conjugate was not observed by surface plasmon resonance analysis (Lee et al., 2016). The Kd of mSAH and biotin was shown to be 7.3×10-10 (Demonte et al., 2013). Although the Kd of mSAH with biotin is higher than that of tetrameric streptavidin, binding of mSAH with biotinylated molecules was shown to be stable more than 1 hour in the presence of competing free biotin.

Pseudotyping lentiviral vectors with biotin-binding envelope proteins.

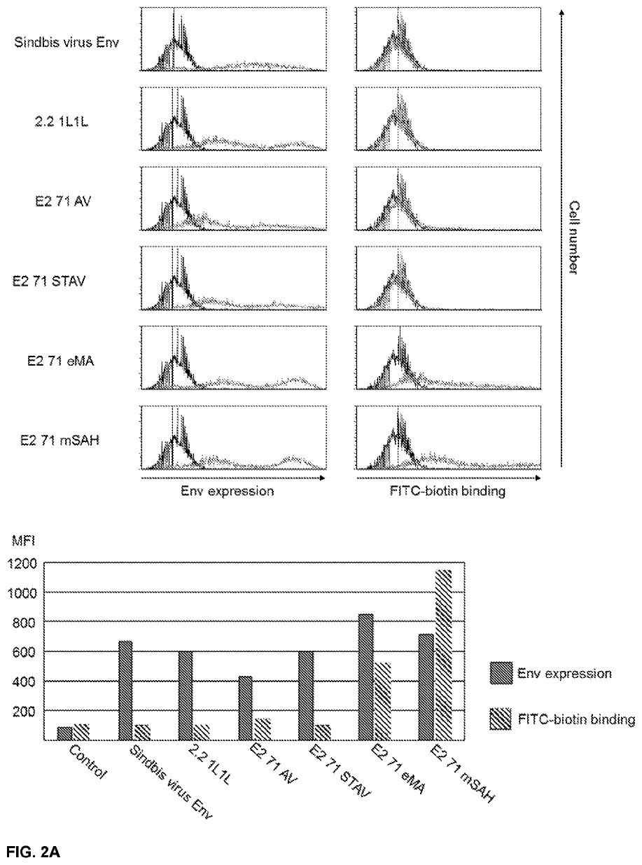

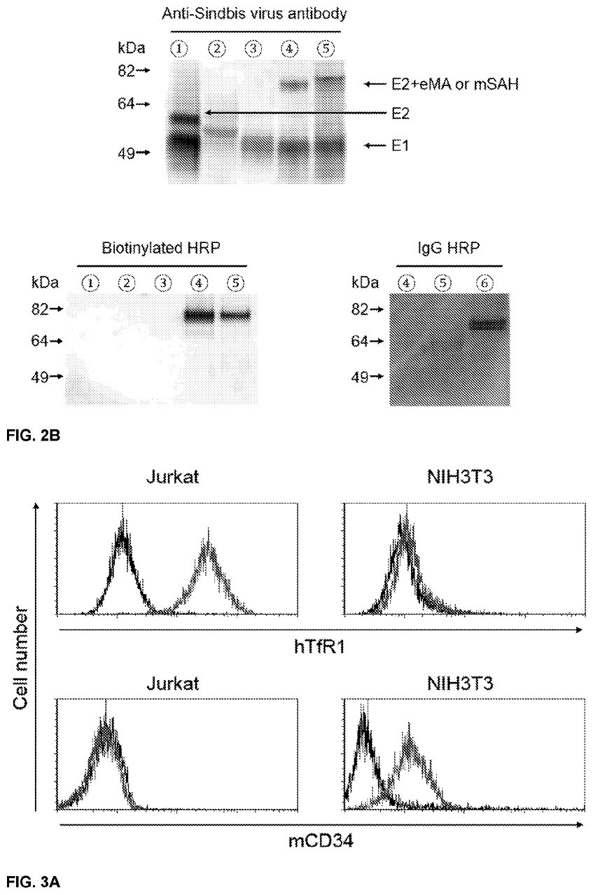

Because pseudotyping of envelope proteins occurs at the surface of vector producer cells, expression of the envelope proteins on the surfaces of transfected 293T cells was first checked. Staining of transfected cells showed that E2 71 AV, E2 71 STAV, E2 71 eMA, and E2 71 mSAH are expressed on the cell surface (FIG. 2 A). To investigate whether these envelope proteins can bind biotinylated molecules, the transfected cells were stained with biotinylated FITC and binding to transfected cells was analyzed by flow cytometry (FIG. 2A). No binding of biotinylated FITC to the cells expressing E2 71 AV or E2 71 STAV was observed, demonstrating that avidin and streptavidin do not have the structures necessary for binding to biotin when fused to the Sindbis virus envelope protein. However, biotinylated FITC bound to cells transfected with E2 71 eMA or E2 71 mSAH.

Also investigated was whether these envelope proteins can pseudotype lentiviral vectors, using western blotting analysis of the purified lentiviral vectors. The fusion envelope proteins were detected using anti-Sindbis virus antibodies and western blot analysis. The molecular weights of the E2 protein of E2 71 eMA were the same as that expected from fusion of the Sindbis virus E2 protein and eMA (˜75 kDa) (FIG. 2B, top). The E2 protein of E2 71 mSAH showed a faint band of the expected size (˜75 kDa) and a strong band at a higher than expected molecular weight (FIG. 2B, top). Because mSAH contains 5 N glycosylation signal sequences (N-X-S/T), it is likely that the higher molecular weight represents N-glycosylation of mSAH. Immunoblot bands were not detected at the expected molecular weights (˜75 kDa) of fusion proteins E2 71 AV and E2 71 STAV, demonstrating that Sindbis virus envelope proteins lose their ability to properly express and/or pseudotype lentiviral vectors when fused to avidin or streptavidin. The cryo-electron microscopic and crystal structure analyses of Sindbis virus envelope proteins previously demonstrated that a heterodimer of E1E2 supports the structure of other heterodimers by forming the trimers of heterodimers (Li et al., 2010; Mukhopadhyay et al., 2006; Pletnev et al., 2001). It is possible that inhibition of the trimerization by avidin and/or streptavidin destabilizes E2 71 AV and E2 71 STAV, so these envelope proteins cannot survive viral purification procedures.

Next investigated was whether the fusion proteins between E2 and eMA or mSAH can specifically bind biotinylated molecules. E2 71 AV and STAV did not show binding of biotinylated HRP by western blot analysis (FIG. 2B, bottom left), which is consistent with lack of binding activity to biotin shown by flow cytometric analysis (FIG. 2A) and low expression on virions (FIG. 2B, top). HRP conjugated with biotin specifically bound to the E2 protein of E2 71 eMA and the lower molecular weight form of E2 of E2 71 mSAH (FIG. 2B, bottom left). HRP conjugated with rabbit IgG specifically bound the E2 fusion protein with ZZ of the 2.2 pseudtotype via the interactions between the IgG Fc and ZZ domains, but did not bind E2 71 eMA or E2 71 mSAH (FIG. 2B, bottom right), demonstrating that binding of HRP-biotin to E2 71 eMA or E2 71 mSAH is biotin-dependent. These results demonstrate that biotinylated molecules can be conjugated to E2 71 eMA and E2 71 mSAH through the interactions between biotin and eMA or mSAH.

Redirecting the E2 71 eMA and E2 71 mSAH pseudotypes by conjugation with biotinylated antibodies.

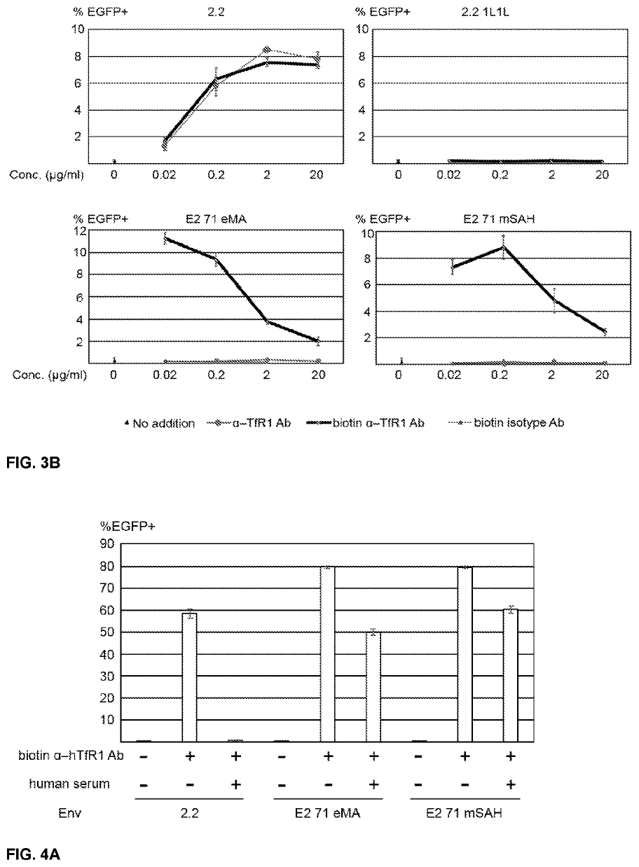

Next investigated was whether the E2 71 eMA and mSAH pseudotypes can transduce cells when conjugated with biotinylated antibodies. Redirection of the tropisms of the pseudotyped vectors was attempted by mixing them with biotinylated or non-biotinylated anti-human TfR1 (hTfR1) antibody and transducing the human T-cell line, Jurkat, which abundantly expresses hTfR1 (FIG. 3A). Conjugation of biotinylated and non-biotinylated anti-hTfR1 antibody with the 2.2 pseudotype drastically increased transduction efficiencies of Jurkat cells with the 2.2 pseudotype. Conjugation of the E2 71 eMA and mSAH pseudotypes with biotinylated anti-hTfR1 antibody but not the non-biotinylated antibody increased the transduction of Jurkat cells. Mixing the 2.2 1L1L pseudotype with biotinylated anti-hTfR1 antibodies did not mediate transduction of Jurkat cells. These results are consistent with the binding specificities of E2 71 eMA and mSAH shown by western blot analysis (FIG. 2B). Conjugation of these pseudotypes with biotinylated isotype control antibody did not mediate transduction of Jurkat cells, demonstrating that binding of conjugated antibodies to target cells is necessary for transduction by E2 71 eMA and mSAH pseudotypes. Although using higher concentrations of viral vectors yields higher percentages of transduction (as shown the in the results of subsequent experiments), the viral amounts that transduce less than 20% of those obtained for the experiments shown in FIG. 3A were used to maintain the linear correlation between the percentages of EGFP+ cells and the titers of the vectors. These results demonstrated that conjugation of targeting antibodies with the E2 71 eMA and mSAH pseudotypes via the interactions between biotin and eMA and mSAH can redirect the tropisms of the vectors.

Of note, while higher concentrations of antibodies do not inhibit antibody-directed transduction of the 2.2 pseudotype, the high concentrations of antibodies decrease transduction of E2 71 eMA and mSAH (FIG. 3B) A research group also reported that targeted lentiviral transduction was inhibited by the presence of excess amounts of targeting ligands when covalently conjugating the targeting ligands to the modified Sindbis virus envelope protein obtained from us (Kasaraneni et al., 2018). It is likely that: 1) excess amounts of the anti-hTFR1 biotinylated antibody competitively bind to hTfR1 expressed on Jurkat cells, thereby inhibiting transduction with the E2 71 eMA and mSAH pseudotypes stably conjugated with anti-hTfR1 antibody; and 2) due to the relatively low affinity of interactions between the ZZ and Fc domains, the 2.2 pseudotype prefers to bind hTfR1 antibodies bound on the surfaces of target cells rather than free excess amounts of the anti-hTFR1 antibody. E2 71 eMA was also conjugated with the lower concentration (0.002 μg/ml) of biotinylated anti-hTfR1 antibody, but the titer is then lower than when conjugated with the same antibody at 0.02 μg/ml.

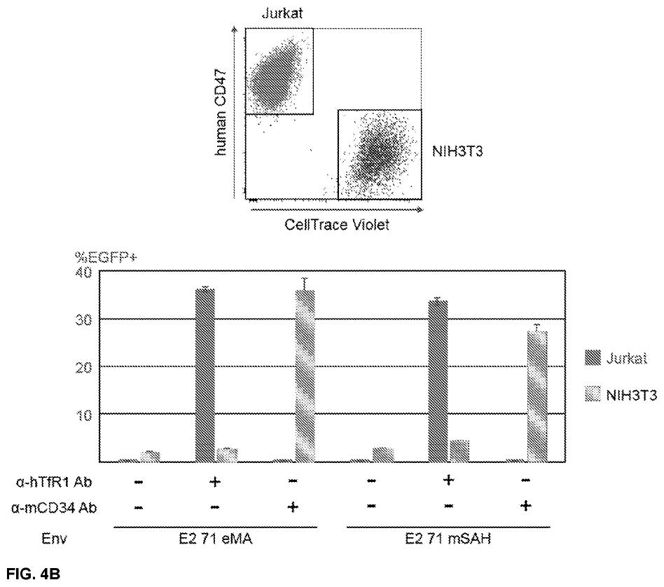

Previous work showed that the antibodies conjugated on 2.2 are detached in the serum due to the competitive binding of serum immunoglobulin to the ZZ domain. To investigate the stability of antibody conjugation, the 2.2, E2 71 eMA, and E2 71 mSAH pseudotypes conjugated with biotinylated anti-hTfR1 antibodies were incubated with human serum before transduction. As previously reported (Morizono et al., 2010), human serum IgG disrupts the conjugation of the antibodies from virus by competitive binding to the ZZ domain and, accordingly, the 2.2 pseudotype conjugated with anti-hTfR1 antibody lost its infectivity (FIG. 4A). On the other hand, E2 71 eMA and mSAH maintain their abilities to transduce Jurkat cells in the presence of human serum, demonstrating more stable conjugation with the antibodies.

To confirm that the E2 71 eMA and mSAH pseudotypes can be redirected according to the specificity of the conjugated antibodies, mixed cultures of two cell types were transduced, using these pseudotypes conjugated with antibodies specific to one cell type in the culture. Anti-mouse CD34 (mCD34) monoclonal antibody binds NIH3T3 but not Jurkat cells (FIG. 3A), whereas anti-hTfR-1 antibody binds Jurkat but not NIH3T3 cells (FIG. 3A). Co-cultures of Jurkat and NIH3T3 cells were transduced with E2 71 eMA or mSAH conjugated with either anti-hTfR1 or mCD34 antibody, and 3 days post-transduction, transgene expression (EGFP) of Jurkat and NIH3T3 cells was analyzed. To distinguish between these two cells types, NIH3T3 was labeled with CellTrace Violet before co-culture, and Jurkat cells were stained with APC-conjugated anti-human CD47 antibody that specifically stains Jurkat cells. EGFP expression of Jurkat (APC+, CellTrace Violet−) and NIH3T3 (APC-, CellTrace Violet+) cells was analyzed individually (FIG. 4B). When the pseudotypes were not conjugated with any antibodies, both cell types were minimally transduced. Conjugation of the pseudotypes with anti-hTfR1 antibody specifically increased transduction of Jurkat cells, and conjugation with anti-mCD34 specifically increased transduction of NIH3T3 cells. These results confirm that the tropisms of the E2 71 eMA and rnSAH pseudotypes are determined by the specificities of the conjugated antibodies.

The effects of biotinylation sites of targeting ligands on the titers of E2 71 eMA and E2 71 mSAH pseudotypes.

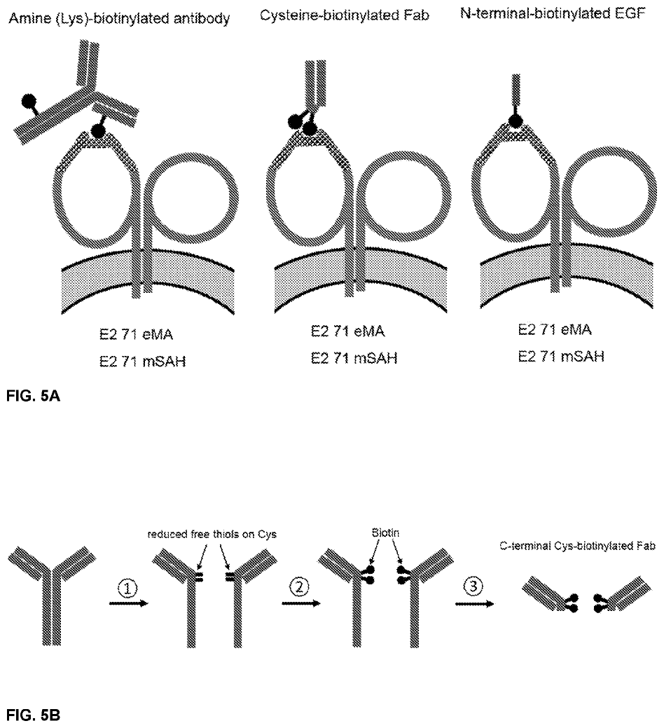

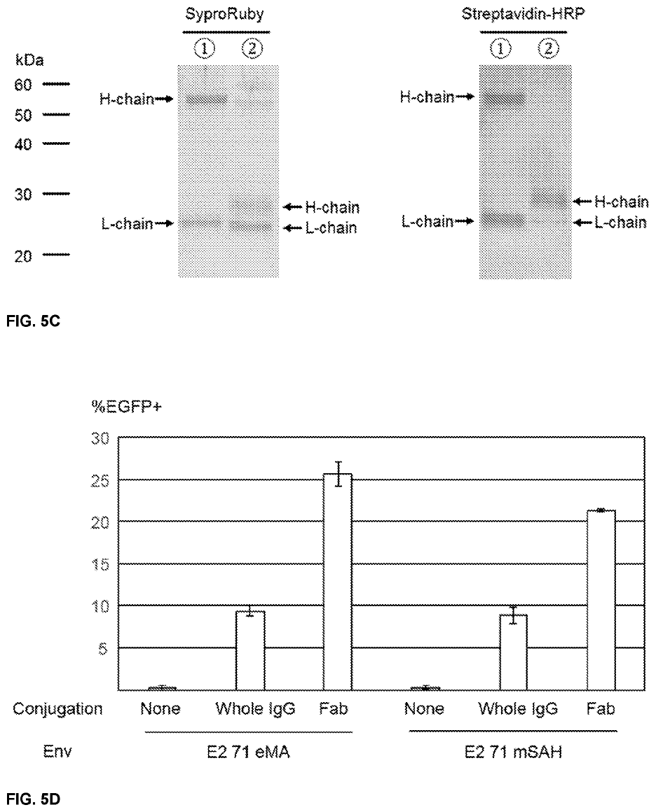

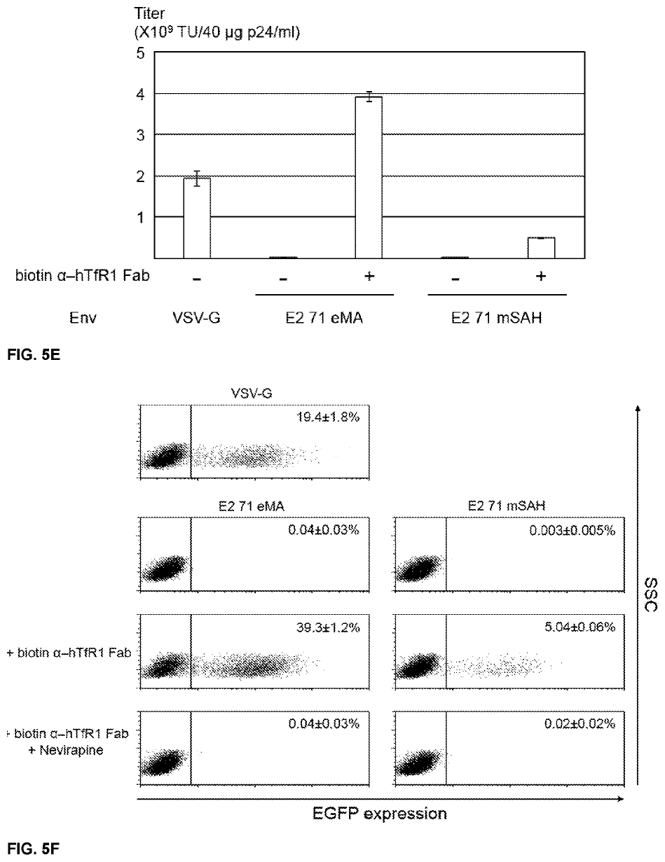

The antibodies against hTfR1 and mCD34 used in the previous experiments were purchased from biotech companies as regular catalog items, Commercial biotinylated antibodies are usually biotinylated by random biotinylation of exposed lysine, using NHS-biotin. Because biotinylation can occur on any exposed lysine, topology of biotinylated antibodies could be in any direction when conjugated with the E2 71 eMA or mSAH pseudotypes (FIG. 5A). If antigen-binding regions of antibodies are not directed toward targeting antigens, the antibody will not be able to efficiently bind to target cells and mediate transduction. Therefore, it is ideal for biotinylation to occur specifically at the C-terminus of antibodies for efficient transduction of target cells with the E2 71 eMA and mSAH pseudotypes (FIG. 5B). For this purpose, specific biotinylation of the C-terminus of the anti-hTfR1 antibody was attempted. The S-S binding was first reduced between heavy chains of anti-hTfR1 antibodies, using β-mercaptoethylamine, followed by biotinylation of the reduced cysteines with Maleimide-PEG2-Biotin (FIG. 5B). To expose conjugated biotin at the C-terminus for better binding to eMA and mSAH, the Fc domain of the antibodies was eliminated by digestion with pepsin (FIG. 5B) and this antibody was designated as biotin anti-hTfR1 Fab. SyproRuby staining showed that the majority of heavy chains were eliminated with biotin anti-hTfR1 Fab (FIG. 5C). Western blotting analysis with streptavidin-HRP showed that biotinylation specifically occurs at heavy chains of biotin anti-hTfR1 Fab, while non-specific biotinylation of lysines biotinylate both light and heavy chains of commercial anti-hTfR 1 antibody. These results demonstrated that biotin anti-hTfR1 Fab possesses biotin at the C-terminus of its heavy chain. Next investigated was whether biotin anti-hTfR1 Fab mediates transduction of the E2 71 eMA and rnSAH pseudotypes more efficiently than biotinylated anti-hTfR1 antibody (FIG. 5D) and found that biotin anti-hTfR1 Fab mediates transduction two-fold more efficiently than biotinylated anti-hTfR1 antibody. These results indicate that controlled biotinylation of the C-terminus of antibodies is ideal for efficient targeting of conjugated vectors.

Transduction of Jurkat cells with the anti-hTfR1 Fab-conjugated E2 71 eMA pseudotype was very efficient, and the titers for the same amount of the viral protein (p24) were higher than with the titers of VSV-G pseudotype (FIG. 5E) [VSV-G: 1.9X109 transduction unit (TU)/40 pg p24/ml, E2 71 eMA +anti-hTfR1 Fab: 3.9X109 TU/40 μg p24/ml]. Since the titers are exceptionally high, it is possible that the EGFP expression of the cells transduced by the E2 71 eMA and SAH pseudotypes is mediated by pseudo transduction, which is the binding of the EGFP protein associated with the vectors (Kim et al., 2017). The flow cytometric profile of EGFP expression from the transduced Jurkat cells showed distinct EGFP-positive populations (FIG. 5F), which indicates endogenous EGFP expression. Further investigated was whether this expression is mediated by the transduced EGFP gene, using the reverse transcriptase inhibitor, nevirapine (Morizono et al., 2010). Nevirapine eliminated more than 99% of EGFP expression of Jurkat cells transduced with anti-hTfR1 Fab conjugated with the E2 71 eMA and mSAH pseudotypes, demonstrating that these vectors induce EGFP expression by transduction.

Redirecting E2 71 eMA and mSAH pseudotypes by biotinylated EGF.

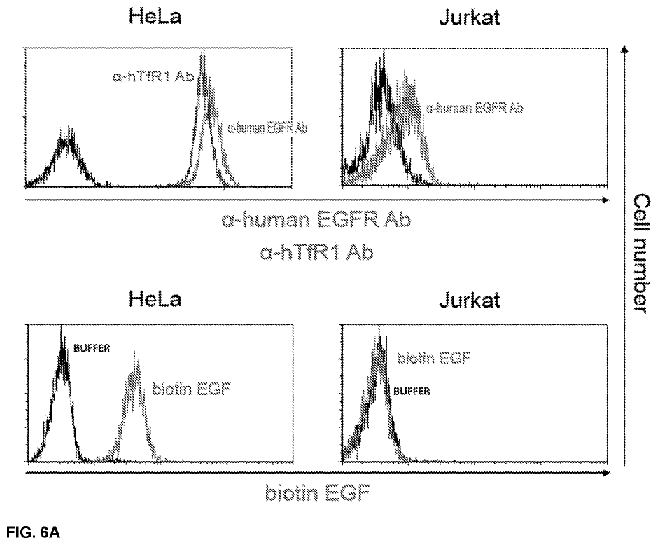

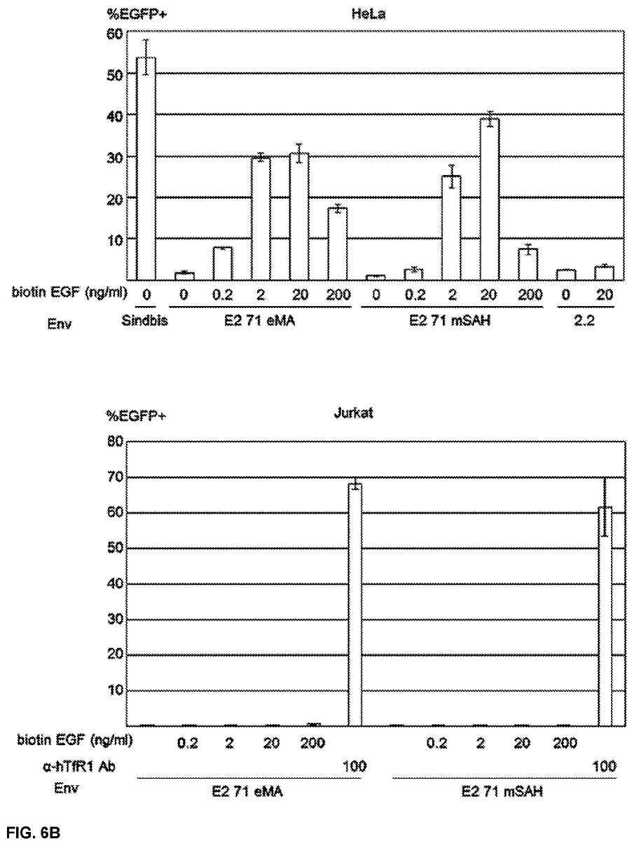

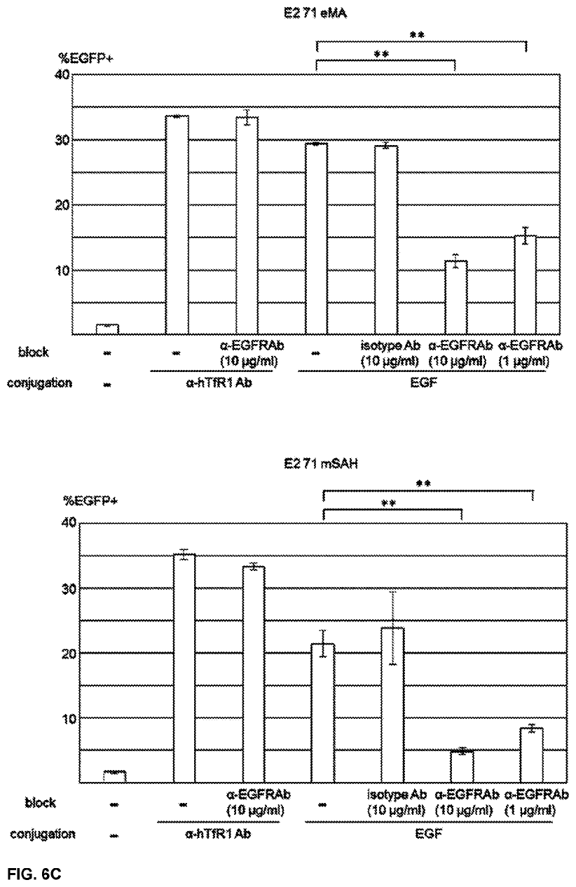

Because conjugation of antibodies to E2 71 eMA and mSAH occurs via the interactions between biotin and eMA and mSAH, the targeting ligand is not limited to antibodies, but occurs with any biotinylated molecule. To test this hypothesis, targeting EGFR was attempted by conjugating biotinylated EGF with the E2 71 eMA and mSAH pseudotypes. HeLa cells abundantly express EGFR, but Jurkat cells do not (FIG. 3A and 6A). Both cell types expressed hTfR1 (FIG. 3A and 6A). HeLa cells can be efficiently transduced by the wild-type Sindbis virus pseudotype. Mutations introduced into its receptor-binding regions decreased the titers of the 2.2, E2 71 eMA and mSAH pseudotypes by more than 30-fold (FIG. 6B). Addition of biotinylated EGF increased transduction of HeLa cells with the E2 71 eMA and mSAH pseudotypes, but not 2.2 (FIG. 6B). Transduction of Hela cells with EGF-conjugated E2 71 eMA and mSAH pseudotypes was blocked by antibodies against EGFR, while transduction of anti-hTfR1 antibody-conjugated E2 71 eMA and mSAH pseudotypes was not (FIG. 6C). Transduction of EGFR-negative Jurkat cells with the E2 71 eMA and mSAH pseudotypes was not enhanced by addition of biotinylated EGFR (FIG. 6B). These results demonstrated that the E2 71 eMA and mSAH pseudotypes can be redirected by any biotinylated molecules.

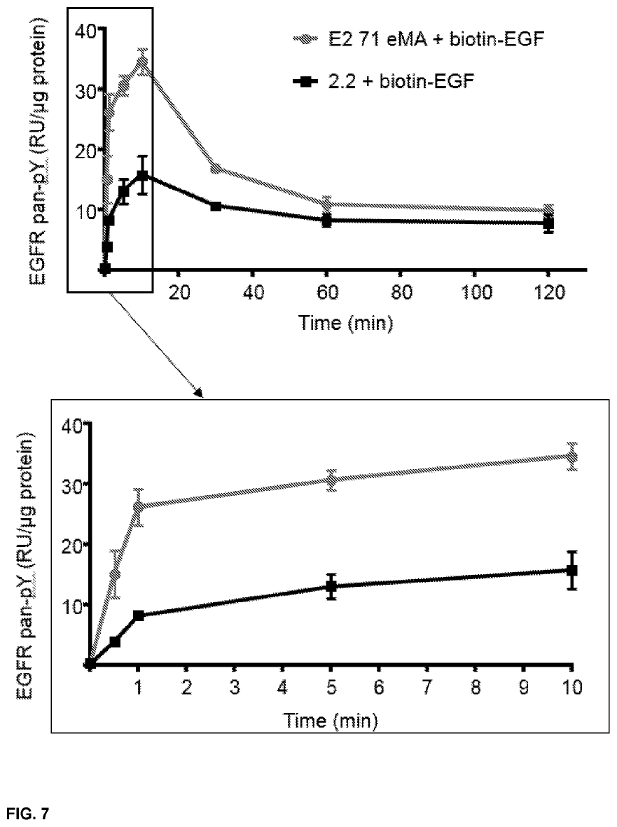

EGF induces signaling via a receptor tyrosine kinase, EGFR, by phosphorylation of the cytoplasmic tyrosine residues of EGFR. It was recently shown that Gas6, a ligand of the TAM family of tyrosine kinase receptors, elicits signaling more efficiently when conjugated on the surface of virions than on free protein (Bhattacharyya et al., 2013). This Example investigated whether EGFR conjugated on virions can induce phosphorylation of EGFR more efficiently than unconjugated (free) EGFR. EGF conjugated with E2 71 eMA pseudotype was added to HeLa cells and phosphorylation of EGFR cytoplasmic tyrosine was analyzed at various time points by Luminex assay. As a control of phosphorylation by free EGF with unconjugated lentiviral vector, the same amount of EGF and 2.2 pseudotype was added. Increases in phosphorylation were observed from 30 sec and peaked at 10 min after addition of EGF (FIG. 7). The EGF mixed with E2 71 eMA induced two-fold more phosphorylation of EGFR than free EGF, which is observed from 30 sec to 2 hours after addition of virus and EGF. These results demonstrated that EGF conjugated on the surface of virus can induce intracellular signaling more efficiently than free EGF.

Discussion

Intensive and time-consuming molecular and/or biochemical manipulations have been required for redirecting lentiviral vectors to individual target molecules (Ahani et al., 2016; Kasaraneni et al., 2017; Kasaraneni et al., 2018; Morizono et al., 2009a) (Morizono et al., 2009b), which has hindered wide applications of targeted lentiviral transduction. This Example reports development of lentiviral vectors that can be stably conjugated with biotinylated targeting molecules and redirected, requiring only simple mixing. Wide varieties of biotinylated antibodies and ligands are commercially available from many manufacturers. For example, the three targeting molecules used in this study, biotinylated anti-hTfR1, mCD34 antibodies, and biotinylated EGF, are regular catalog products of globally accessible manufacturers (Biolegend and ThermoFisher Scientific) In addition, conjugating biotin to lysine residues is a relatively simple procedure that does not require special devices or techniques; thus, researchers can biotinylate targeting ligands of interest if they are not commercially available.

The highly stable binding of this targeting technology will be important for various in vitro and in vivo settings. The conjugation method involves simply mixing virus and biotinylated targeting ligands, and the availability of wide varieties of biotinylated molecules from various companies enables many researchers to utilize this technology to target cells of interest.

Fusion of the E2 protein with wild-type avidin and streptavidin interfered with the ability of the envelope to pesudotype lentiviral vectors and of avidin and streptavidin to bind biotin. It is likely that tetramerization induced by wild-type avidin and streptavidin interferes with trimerization of Sindbis virus envelope proteins, which is required for proper folding and expression on the envelope. Development of monomeric rhizavidin and streptavidin, which bind biotin at high affinities, enabled us to express biotin-binding envelope proteins on the envelope. Viral envelope proteins of various viruses are known to form trimers (Fields, Knipe, and Howley, 2013) (Gibbons et al., 2000; Wilson, Skehel, and Wiley, 1981; Zhu et al., 2006). eMA and mSAH may be able to fuse with such envelope proteins without interfering with the expression and functions of the envelope proteins. In addition, receptor-binding proteins of non-enveloped viruses such as adenovirus also form timers (Xia et al., 1994). Fusion of these proteins with eMA and/or mSAH may facilitate redirection of the tropisms of non-envelope viral vectors by conjugation with biotinylated ligands.

The results comparing non-specific lysine and site-specific biotinylation indicate that the topology of conjugated antibodies is important for efficient transduction of target cells. The placement of targeting ligands on the viral surface can adversely affect the antigen-binding regions of these ligands, In the case of redirecting measles virus envelope proteins fused with targeting ligands, linkers between targeting ligands, and envelope proteins resulted in reduced titers of the pseudotyped lentiviral vectors (Rasbach et al., 2013).

The importance of the topology of conjugated targeting ligands will be dependent on how the ligands bind to target antigens. A previous study by another research group showed that the targeting ligand conjugated on the virus cannot efficiently bind the membrane proximal site of the HER2/nre receptor, while the conjugated ligand targeting the membrane distal sites with the same receptor can do so efficiently (Kasaraneni et al., 2018) . If the binding sites of the ligands are localized to the exposed uppermost surface of targeted molecules, the topology of the targeting ligands conjugated on the envelope may not affect binding and transduction efficiencies of vectors. When the binding site is located at the plasma membrane-proximal sites, it is likely that the targeting ligands must be directed towards the binding site to optimally access and bind targeted cell surface molecules.

The results showed that intracellular EGFR signaling was triggered by the binding of the conjugated vectors to the surface receptor. As previously reported, Gas6, which binds the viral envelope via binding to the envelope lipid, phosphatidylserine (PtdSer), drastically enhanced lentiviral transduction and infection of replication-competent vaccinia virus by 10-50-fold

(Morizono and Chen, 2014; Morizono et al., 2011). Subsequent studies of other groups showed that 80% of this enhancement is mediated by signaling via the binding of Gas6 to its receptors (Bhattacharyya et al., 2013). While binding of Gas6 to virus is specifically mediated by the PtdSer-binding region of Gas6 and PtdSer of the viral envelope, E2 71 eMA and mSAH will enable the display of any biotinylated signaling molecule.

Antibodies against CD3 fused with GPI-anchors were previously displayed on the viral membrane to induce signaling in T cells (Derdak et al., 2006). Signaling via CD3 is known to facilitate a post-binding step(s) of lentiviral transduction and HIV infection (Korin and Zack, 1998; Zack et al., 1990). While these studies used displayed ligands on vector particles for the purpose of cell activation and not for targeting, the system allows for targeted transduction that is facilitated by both specific binding and induction of supportive signaling for lentiviral transduction. Because any type of biotinylated molecule can be used as a conjugating ligand, this technology will enable researchers in various research fields to transduce target cells and/or elicit signaling, using a wide variety of targeting and/or signaling ligands.

The previous targeting lentiviral vector system with the ZZ domain has been used previously for specific transduction of desired cell types in immunodeficient mice (Lafitte et al., 2012; Morizono et al., 2005; Pariente et al., 2007; Zhang et al., 2011; Zhang et al., 2009), demonstrating the proof of principle of the targeted lentiviral transduction. High stability of binding between E2 71 eMA and mSAH and biotinylated ligands will enable this targeting lentiviral transduction system to be applicable to immunocompetent animal experimental settings, facilitating application of targeted lentiviral transduction to broad fields of research.

### REFERENCES

Ahani, R., et al. (2016). Sindbis Virus-Pseudotyped Lentiviral Vectors Carrying VEGFR2-Specific Nanobody for Potential Transductional Targeting of Tumor Vasculature. Molecular biotechnology 58(11), 738-747.

Aires da Silva, F., et al. (2005). Cell type-specific targeting with sindbis pseudotyped lentiviral vectors displaying anti-CCR5 single-chain antibodies. Hum Gene Ther 16(2), 223-34.

Anderson, J. S., et al. (2009). Specific transduction of HIV-susceptible cells for CCR5 knockdown and resistance to HIV infection: a novel method for targeted gene therapy and intracellular immunization. Journal of acquired immune deficiency syndromes 52(2), 152-61.

Bayer, E. A., and Wilchek, M. (1990). Biotin-binding proteins: overview and prospects. Methods in enzymology 184, 49-51.

Bender, R. R., et al. (2016). Receptor-Targeted Nipah Virus Glycoproteins Improve Cell-Type Selective Gene Delivery and Reveal a Preference for Membrane-Proximal Cell Attachment. PLoS pathogens 12(6), e1005641.

Bergman, I., et al. (2004). Preferential targeting of vesicular stomatitis virus to breast cancer cells. Virology 330(1), 24-33.

Bhattacharyya, S., et al. (2013). Enveloped viruses disable innate immune responses in dendritic cells by direct activation of TAM receptors. Cell host & microbe 14(2), 136-47.

Brown, B. D., et al. (2006). Endogenous microRNA regulation suppresses transgene expression in hematopoietic lineages and enables stable gene transfer. Nat Med 12(5), 585-91.

Cao, Z., et al. (2016). Targeting of the pulmonary capillary vascular niche promotes lung alveolar repair and ameliorates fibrosis. Nat Med 22(2), 154-62.

Cavazzana-Calvo, M., et al. (2010). Transfusion independence and HMGA2 activation after gene therapy of human beta-thalassaemia. Nature 467(7313), 318-22.

Demonte, D., et al. (2013). Structure-based engineering of streptavidin monomer with a reduced biotin dissociation rate. Proteins 81(9), 1621-33.

Derdak, S. V., et al. (2006). Direct stimulation of T lymphocytes by immunosomes:

virus-like particles decorated with T cell receptor/CD3 ligands plus costimulatory molecules. Proc Nati Acad Sci USA 103(35), 13144-9.

Dubuisson, J., and Rice, C. M. (1993). Sindbis virus attachment: isolation and characterization of mutants with impaired binding to vertebrate cells, J Virol 67(6), 3363-74.

Fielding, A. K., et al. (1998). Inverse targeting of retroviral vectors: selective gene transfer in a mixed population of hematopoietic and nonhematopoietic cells. Blood 91(5), 1802-9.

Fields, B. N., et al. (2013). “Fields virology.” 6th ed. 2 vols. Wolters Kluwer Health/Lippincott Williams & Wilkins, Philadelphia.

Fraietta, J. A., et al. (2018). Disruption of TET2 promotes the therapeutic efficacy of 0019-targeted T cells. Nature 558(7709), 307-312.

Funke, S., et al. (2008). Targeted cell entry of lentiviral vectors. Mol Ther 16(8), 1427-36.

Gibbons, D. L., et al. (2000). Formation and characterization of the trimericorm of the fusion protein of Semliki Forest Virus. Journal of virology 74(17), 7772-80.

Helppolainen, S. H. et al. (2007) Rhizavidin from Rhizobium etli: the first natural dimer in the avidin protein family. The Biochemical journal 405(3), 397-405.

Huhtala, T., et al. (2014). Biodistribution and antitumor effect of Cetuximab-targeted lentivirus. Nucl Med Biol 41(1), 77-83.

Kaikkonen, M. U., et al. (2009). (Strept)avidin-displaying lentiviruses as versatile tools for targeting and dual imaging of gene delivery. Gene Ther 16(7), 894-904.

Kasahara, N., et al. (1994). Tissue-specific targeting of retroviral vectors through ligand-receptor interactions. Science 266(5189), 1373-6.

Kasaraneni, N., et al. (2017). Retargeting Lentiviruses via SpyCatcher-SpyTag Chemistry for Gene Delivery into Specific Cell Types. MBio 8(6).

Kasaraneni, N., et al. (2018). A simple strategy for retargeting lentiviral vectors to desired cell types via a disulfide-bond-forming protein-peptide pair. Sci Rep 8(1), 10990.

Kim, J. T., et al. (2017). Dendritic cell-targeted lentiviral vector immunization uses pseudotransduction and DNA-mediated STING and cGAS activation. Sci Immunol 2(13).

Klimstra, W. B., et al. (1999). The furin protease cleavage recognition sequence of Sindbis virus PE2 can mediate virion attachment to cell surface heparan sulfate. J Virol 73(8), 6299-306.

Korin, Y. D., and Zack, J. A. (1998). Progression to the G1b phase of the cell cycle is required for completion of human immunodeficiency virus type 1 reverse transcription in T cells. J Virol 72(4), 3161-8.

Lafitte, M., et al. (2012). In vivo gene transfer targeting in pancreatic adenocarcinoma with cell surface antigens. Mol Cancer 11, 81.

Laitinen, O. H., et al. (2006). Genetically engineered avidins and streptavidins. Cell Mol Life Sci 63(24), 2992-3017.

Lee, J. M., et al. (2016). A Rhizavidin Monomer with Nearly Multimeric Avidin-Like Binding Stability Against Biotin Conjugates, Angewandte Chemie 55(10), 3393-7.

Li, L., et al. (2010). Structural changes of envelope proteins during alphavirus fusion. Nature 468(7324), 705-8.

Liang, M., et al. (2009a). Targeted transduction via CD4 by a lentiviral vector uses a clathrin-mediated entry pathway. J Virol 83(24), 13026-31.

Liang, M., et al. (2009b). Targeted transduction of CD34+hematopoietic progenitor cells in nonpurified human mobilized peripheral blood mononuclear cells. J Gene Med 11(3), 185-96.

Morizono, K., et al. (2001). Antibody-directed targeting of retroviral vectors via cell surface antigens. J Virol 75(17), 8016-20.

Morizono, K., and Chen, I. S. (2005). Targeted gene delivery by intravenous injection of retroviral vectors. Cell Cycle 4(7), 854-6.

Morizono, K., and Chen, I. S. (2011). Receptors and tropisms of envelope viruses. Current opinion in virology 1(1), 13-18.

Morizono, K., and Chen, I. S. (2014). Role of phosphatidylserine receptors in enveloped virus infection. Journal of virology 88(8), 4275-90.

Morizono, K., et al. (2010). Redirecting lentiviral vectors pseudotyped with Sindbis virus-derived envelope proteins to DC-SIGN by modification of N-linked glycans of envelope proteins.

J Virol 84(14), 6923-34.

Morizono, K., et al. (2009a). Redirecting lentiviral vectors by insertion of integrin-tageting peptides into envelope proteins. J Gene Med 11(7), 549-58.

Morizono, K., et al. (2006). Transient low pH treatment enhances infection of lentiviral vector pseudotypes with a targeting Sindbis envelope. Virology 355(1), 71-81.

Morizono, K., et al. (2009b). A versatile targeting system with lentiviral vectors bearing the biotin-adaptor peptide. J Gene Med 11(8), 655-63.

Morizono, K., et al. (2011). The Soluble Serum Protein Gas6 Bridges Virion Envelope Phosphatidylserine to the TAM Receptor Tyrosine Kinase Axl to Mediate Viral Entry. Cell Host Microbe 9(4), 286-98.

Morizono, K., et al. (2005). Lentiviral vector retargeting to P-glycoprotein on metastatic melanoma through intravenous injection. Nat Med 11(3), 346-52.

Mukhopadhyay, S., et al. (2006). Mapping the structure and function of the E1and E2 glycoproteins in alphaviruses. Structure 14(1), 63-73.

Munch, R. C., et al. (2011). DARPins: An Efficient Targeting Domain for Lentiviral Vectors. Mol Ther.

Nakamura, T., et al. (2005). Rescue and propagation of fully retargeted oncolytic measles viruses. Nat Biotechnol 23(2), 209-14.

Naldini, L., et al. (1996a). Efficient transfer, integration, and sustained long-term expression of the transgene in adult rat brains injected with a lentiviral vector. Proc Natl Acad Sci USA 93(21), 11382-8.

Naldini, L., et al. (1996b) In vivo gene delivery and stable transduction of nondividing cells by a lentiviral vector. Science 272(5259), 263-7.

Naldini, L., et al. (2016). Lentiviral vectors, two decades later. Science 353(6304), 1101-2.

Ohno, K., et al. (1997). Cell-specific targeting of Sindbis virus vectors displaying lgG-binding domains of protein A. Nat Biotechnol 15(8), 763-7.

Pariente, N., et al. (2008). Efficient targeted transduction of primary human endothelial cells with dual-targeted lentiviral vectors. J Gene Med 10(3), 242-8.

Pariente, N., et al. (2007). A novel dual-targeted lentiviral vector leads to specific transduction of prostate cancer bone metastases in vivo after systemic administration. Mol Ther 15(11), 1973-81.

Pletnev, S. V., et al. (2001). Locations of carbohydrate sites on alphavirus glycoproteins show that E1forms an icosahedral scaffold. Cell 105(1), 127-36.

Ranzani, M., et al. (2013). Cancer gene discovery: exploiting insertional mutagenesis. Mol Cancer Res 11(10), 1141-58.

Rasbach, A., et al. (2013). The receptor attachment function of measles virus hemagglutinin can be replaced with an autonomous protein that binds Her2/neu while maintaining its fusion-helper function. J Virol 87(11), 6246-56.

Sandrin, V., et al. (2003). Targeting retroviral and lentiviral vectors. Curr Top Microbiol Immunol 281, 137-78.

Sessa, M., et al. (2016). Lentiviral haemopoietic stem-cell gene therapy in early-onset metachromatic leukodystrophy: an ad-hoc analysis of a non-randomised, open-label, phase 1/2 trial. Lancet 388(10043), 476-87.

Somia, N. V., et al. (1995). Generation of targeted retroviral vectors by using single-chain variable fragment: an approach to in vivo gene delivery, Proc Natl Aced Sci USA 92(16), 7570-4.

Wilson, A. A., et al. (2010). Amelioration of emphysema in mice through lentiviral transduction of long-lived pulmonary alveolar macrophages. J Clin Invest 120(1), 379-89.

Wilson, I. A., et al. (1981). Structure of the haemagglutinin membrane glycoprotein of influenza virus at 3 A resolution. Nature 289(5796), 366-73.

Wu, D. T., et al. (2012). Antibody-directed lentiviral gene transduction for live-cell monitoring and selection of human iPS and hES cells. PloS one 7(4), e34778.

Xia, D., et al. (1994). Crystal structure of the receptor-binding domain of adenovirus type 5 fiber protein at 1.7 A resolution. Structure 2(12), 1259-70.

Yang, L., et al. (2006). Targeting lentiviral vectors to specific cell types in vivo. Proc Natl Acad Sci USA 103(31), 11479-84.

Zack, J. A., et al. (1990). HIV-1 entry into quiescent primary lymphocytes: molecular analysis reveals a labile, latent viral structure. Cell 61(2), 213-22.

Zhang, K. X., et al. (2011). Targeting trastuzumab-resistant breast cancer cells with a lentivirus engineered to bind antibodies that recognize HER-2. Breast Cancer Res Treat 125(1), 89-97.

Zhang, K. X., et al. (2009). Lentiviruses with trastuzumab bound to their envelopes can target and kill prostate cancer cells. Cancer gene therapy 16(11), 820-31.

Zhang, X., and Roth, M. J. (2010). Antibody-directed lentiviral gene transduction in early immature hematopoietic progenitor cells. The journal of gene medicine 12(12), 945-55.

Zhao, Y., et al. (1999). Identification of the block in targeted retroviral-mediated gene transfer. Proc Natl Acad Sci USA 96(7), 4005-10.

Zhu, P., et al. (2006). Distribution and three-dimensional structure of AIDS virus envelope spikes. Nature 441(7095), 847-52.

### Example 2

### Transduction of B-cells by Intravenous Administration of Targeting Vector

Example 1 shows that the targeting lentiviral vector minimally transduces untargeted organs when examined in immunodeficient mice. The remaining low-level non-specific transduction occurs in the liver and spleen. However, most of the immune cell types are lacking because immunodificient mice lack normal development of hematopoietic cells, especially the cell lineages that have important roles in immunity. Since immune cells are known to trap pathogens by special molecular mechanisms (Morizono et.al, Cell Host and Microbe 2011, Morizono etal., Journal of Virology 2014), whether the targeting vector non-specifically transduces untargeted organs without conjugation of targeting ligands was next investigated.

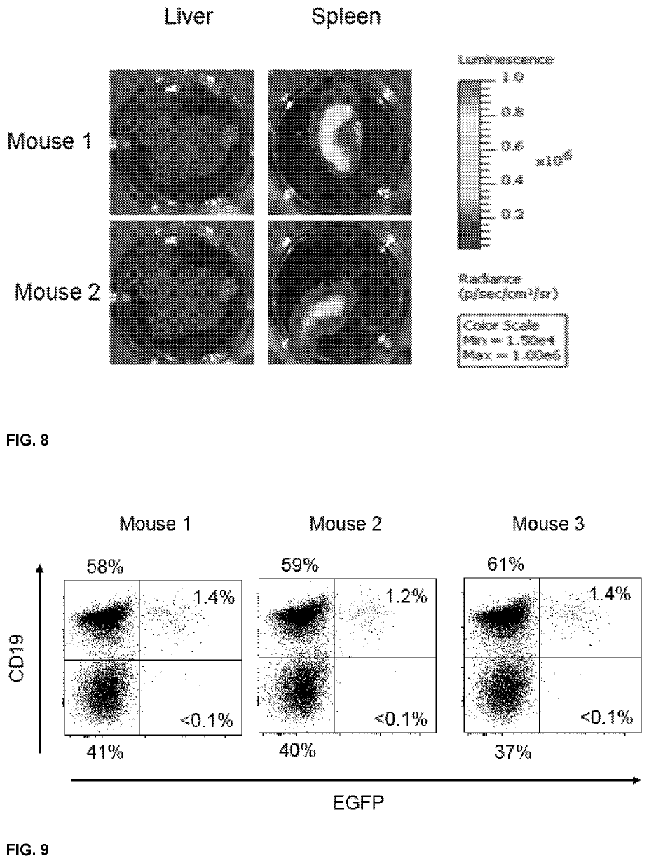

Firefly luciferase was used as the transgene of the lentiviral vector. The vector was intravenously injected into immunocompetent mice (C57BL6). Transgene (luciferase) expression was monitored 5 days after injection. As shown in FIG. 8, transduction of the liver is still minimal (just above detection threshold). Strong transgene expression was observed from the spleen, which was not observed with immunodeficient mice. Unlike immunodeficient mice, the spleen of immunocompetent mice has many types of immune cells. Thus, it is likely that the significant transduction observed in the spleen occurs in certain types of immune cells that are absent in immunodeficient mice. Of note, transgene expression from blood cells was not observed. Next investigated was which cell types in the spleen are transduced by the targeting lentiviral vector without conjugation of targeting ligands.

The vector harboring EGFP as its transgene was injected intravenously into immunocompetent mice (C57BL6) and transgene (EGFP) expression was analyzed at the splenic cells 5 days after injection. The splenic cells were isolated, and stained with cell surface markers of immune cells. EGFP expression in each immune cells types was analyzed by flow cytometry. As shown in FIG. 9, transgene (EGFP) is manly expressed in CD19+ is the definitive cell surface marker of B-cells, which are not present in immunodeficient mice.

Although specific and efficient transduction of B-cells by the vector is unexpected, B-cell transduction offers several advantages. One is that B-cells are known to induce tolerance to transgene products expressed inside B-cells (Wang, X., et al. Mol Ther Methods Clin Dev 2017, Skupsky, J. et al. Molecular Therapy 2010, Su, Y., Frontier Microbiology, 2011). The other is that certain subpopulations of B-cells, such as long-lived plasma cells can live decades, which enables lentiviral transgene expression for long periods. Thus the investigation turned to identification of the subpopulation of B-cells transduced by staining EGFP-transduced cells with various B-cell subpopulation markers.

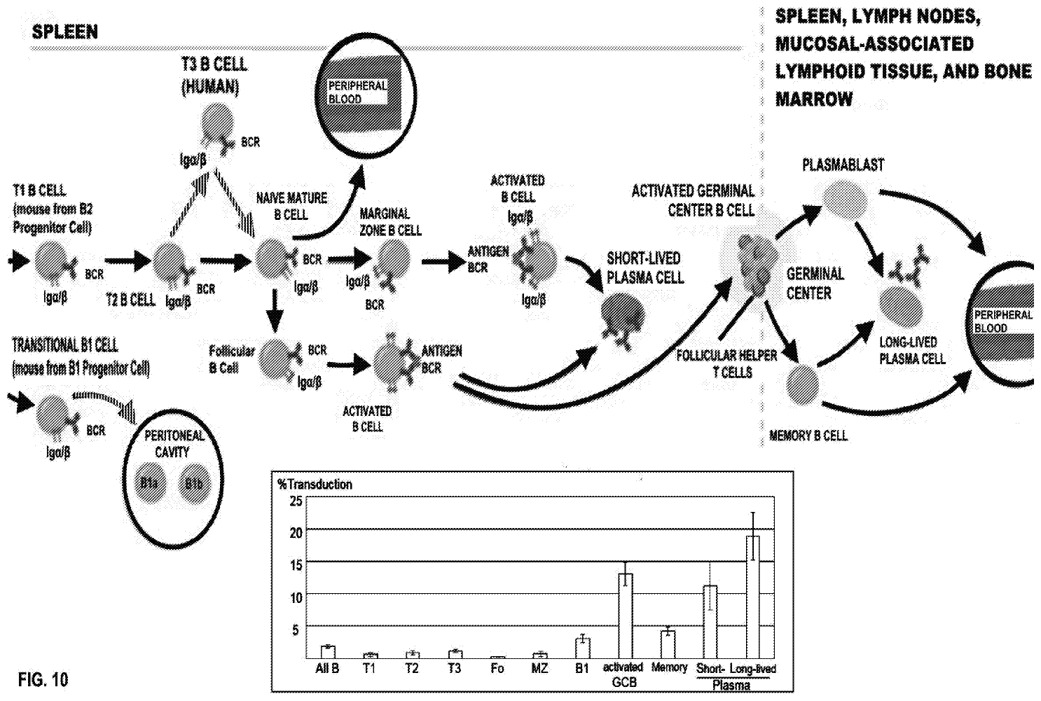

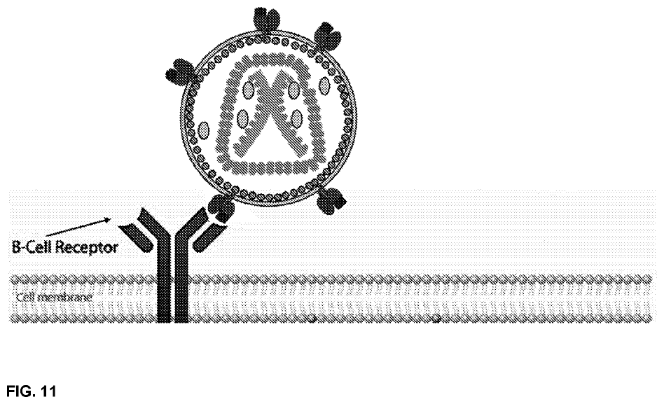

EGFP-transduced cells were stained with markers of various B-cell subpopulations shown in FIG. 10. Flow cytometry was used to analyze what B-cell subpopulation is expressing EGFP. The cell type most efficiently transduced is long-lived plasma cells. These results indicate that the specific transduction of this population by the targeting vector can be used for long-term transgene expression due to the ability of B-cells to induce tolerance to transgene products and long life span of long-lived plasma cells.

Throughout this application various publications and patents are referenced. The disclosures of these publications and patents in their entireties are hereby incorporated by reference into this application in order to describe more fully the state of the art to which this invention pertains.

Those skilled in the art will appreciate that the conceptions and specific embodiments disclosed in the foregoing description may be readily utilized as a basis for modifying or designing other embodiments for carrying out the same purposes of the present invention. Those skilled in the art will also appreciate that such equivalent embodiments do not depart from the spirit and scope of the invention as set forth in the appended claims

1. A targeting construct comprising a nucleic acid sequence encoding a modified Sindbis virus envelope protein fused with a monomeric biotin-binding molecule, wherein the modified Sindbis virus envelope protein comprises mutant E2 and E3 proteins.

2. The targeting construct of claim 1, wherein the mutant E2 protein lacks the sequence SLKQ, and wherein the mutant E3 protein lacks the sequence RSKR.

3. The targeting construct of claim 1, wherein the monomeric biotin-binding molecule is rhizavidin or a rhizavidin/streptavidin hybrid.

4. The targeting construct of claim 3, wherein the mutant E2 protein is E2 71 eMA or E2 71 mSAH.

5. The targeting construct of claim 1, further comprising a biotinylated targeting ligand conjugated with the biotin-binding molecule.

6. The targeting construct of claim 5, wherein the targeting ligand is an antibody.

7. The targeting construct of claim 5, wherein the targeting ligand is a receptor ligand.

8. A pseudotyped retrovirus vector comprising the targeting construct of claim 1, and, optionally, a heterologous gene.

9. A method of transducing a target cell with a heterologous gene, the method comprising contacting the target cell with a pseudotyped retrovirus vector of claim 8.

10. The method of claim 9, wherein the contacting occurs in vitro.

11. The method of claim 9, wherein the contacting occurs ex vivo.

12. The method of claim 9, wherein the contacting occurs in vivo.

13. The method of claim 9, wherein the target cell is a B cell.

14. The method of claim 9, wherein the target cell is a T cell.

15. The method of claim 9, wherein the heterologous gene encodes a chimeric antigen receptor.

16. The method of claims 17, wherein the targeting ligand is an antibody that specifically binds CD3, Interlukin-7, protein L, CD40, and/or a B-cell receptor.

17. The method of claim 9, wherein the targeting construct further comprises a biotinylated targeting ligand conjugated with the biotin-binding molecule.
